SEQUENCE LISTING (33 sequences)

SEQ ID NO: 1 - DNA, 711 bp, Homo sapiens
  CDS (8)..(547)
cagcaagatggtgttgcagacccaggtcttcatttctctgttgctctggatctctggtgcctacggggacatcgtgatgacccagtctccagactccctggctgtgtctctgggcgagagggccaccatcaactgcaagtccagccagagtgttttatacagctccaacaataagaactacttagcttggtaccagcagaaaccaggacagcctcctaagctgctcatttactgggcatctacccgggaatccggggtccctgaccgattcagtggcagcgggtctgggacagatttcactctcaccatcagcagcctgcaggctgaagatgtggcagtttattactgtcagcaatattatagtactcctcccacagtgcttcagcctcgaacacaaacctcctccccatacgctgggccagtaggtctttgctgcagcagctgcttcctctgcacacagcccccaacatgcatgcttcctctgtgtgttggggaggtcactctcttgatttattcgttggagggtttgcagggcccaggattaaattaagagacttgacttttgctggatctctttttgtagaagattattaaagcaaaatgttgtaaagatcccttagagacattgtcaggagtttttgtgttacaggaacctgcatgtttcacatggacacatcacatgaccgagccaaatagatttatctttactct

SEQ ID NO: 2 - protein, 180 aa, Homo sapiens
MVLQTQVFISLLLWISGAYGDIVMTQSPDSLAVSLGERATINCKSSQSVLYSSNNKNYLAWYQQKPGQPPKLLIYWASTRESGVPDRFSGSGSGTDFTLTISSLQAEDVAVYYCQQYYSTPPTVLQPRTQTSSPYAGPVGLCCSSCFLCTQPPTCMLPLCVGEVTLLIYSLEGLQGPGLN

SEQ ID NO: 3 - DNA, 408 bp, Homo sapiens
  CDS (1)..(405)
gtgagaagggtttttgttcagcaagacaatggagagctcacactgtggtggacgttcggccaagggaccaaggtggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaactctacgcctgcgaagtcacccatcagggcctgagctcgcccgtcacaaagagcttcaacaggggagagtgttag

SEQ ID NO: 4 - protein, 135 aa, Homo sapiens
VRRVFVQQDNGELTLWWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKLYACEVTHQGLSSPVTKSFNRGEC

SEQ ID NO: 5 - protein, 121 aa, Homo sapiens
MVLQTQVFISLLLWISGAYGDIVMTQSPDSLAVSLGERATINCKSSQSVLYSSNNKNYLAWYQQKPGQPPKLLIYWASTRESGVPDRFSGSGSGTDFTLTISSLQAEDVAVYYCQQYYSTP

SEQ ID NO: 6 - protein, 107 aa, Homo sapiens [sequence omitted]

SEQ ID NO: 7 - protein, 103 aa, Homo sapiens
DIQMTQSPSSVSASVGDRVTITCRASQGISSWLAWYQQKPGKAPKLLIYAASSLQSGVPSRFSGSGSGTDFTLTISSLQPEDFATYYCQQANSFPPTVLPTRT

SEQ ID NO: 8 - protein, 103 aa, Homo sapiens
DIQMTQSPSSLSASVGDRVTITCRASQGISNYLAWFQQKPGKAPKSLIYAASSLQSGVPSRFSGSGSGTDFTLTISSLQPEDFATYYCQQYNSYPPTVLHTQT

SEQ ID NO: 9 - protein, 110 aa, Homo sapiens
DIQMTQSPSSLSASVGDRVTITCRASQGIRNDLGWYQQKPGKAPKRLIYAASSLQSGVPSRFSGSGSGTEFTLTISSLQPEDFATYYCLQHNSYPPTVLHTRTTPREADV

SEQ ID NO: 10 - protein, 98 aa, Homo sapiens
DIQMTQSPSSLSASVGDRVTITCQASQDISNYLNWYQQKPGKAPKLLIYDASNLETGVPSRFSGSGSGTDFTFTISSLQPEDIATYYCQQYDNLPPTV

SEQ ID NO: 11 - protein, 101 aa, Homo sapiens
DIQMIQSPSFLSASVGDRVSIICWASEGISSNLAWYLQKPGKSPKLFLYDAKDLHPGVSSRFSGRGSGTDFTLTIISLKPEDFAAYYCKQDFSYPPTGLQA

SEQ ID NO: 12 - protein, 135 aa, Homo sapiens
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPPTVVQPLTETSSWGCPVAHMCCLSGEQLSRVSESA

SEQ ID NO: 13 - protein, 106 aa, Homo sapiens
DVVMTQSPLSLPVTLGQPASISCRSSQSLVYSDGNTYLNWFQQRPGQSPRRLIYKVSNRDSGVPDRFSGSGSGTDFTLKISRVEAEDVGVYYCMQGTHWPPTVVQP

SEQ ID NO: 14 - protein, 132 aa, Homo sapiens
EIVLTQSPATLSLSPGERATLSCRASQSVSSYLAWYQQKPGQAPRLLIYDASNRATGIPARFSGSGSGTDFTLTISSLEPEDFAVYYCQQRSNWPPTVIPHETKTPTRPSVFTRLLYQLLPLQTASGVATQC

SEQ ID NO: 15 - protein, 118 aa, Homo sapiens
EIVLTQSPATLSLSPGERATLSCRASQGVSSYLAWYQQKPGQAPRLLIYDASNRATGIPARFSGSGPGTDFTLTISSLEPEDFAVYYCQQRSNWHPTVIPHETKTPTRPSVFTRLLYQ

SEQ ID NO: 16 - protein, 115 aa, Homo sapiens
ETTLTQSPAFMSATPGDKVNISCKASQDIDDDMNWYQQKPGEAAIFIIQEATTLVPGIPPRFSGSGYGTDFTLTINNIESEDAAYYFCLQHDNFPLTVIHPVQKPPSSLSGIASA

SEQ ID NO: 17 - protein, 158 aa, Homo sapiens
DVVMTQSPAFLSVTPGEKVTITCQASEGIGNYLYWYQQKPDQAPKLLIKYASQSISGVPSRFSGSGSGTDFTFTISSLEAEDAATYYCQQGNKHPHTVLQPKTKISSAWRNRETEQYPVFMILAGAVGEIIYQIPSHMAHSAELTPKSQCLTLSSLPT

SEQ ID NO: 18 - protein, 103 aa, Homo sapiens
DIVLTQSPASLAVSPGQRATITCRASESVSFLGINLIHWYQQKPGQPPKLLIYQASNKDTGVPARFSGSGSGTDFTLTINPVEANDTANYYCLQSKNFPPTVL

SEQ ID NO: 19 - protein, 119 aa, Homo sapiens
WTFGQGTKVEIKRTVAAPSVFIFPPSDEQLKSGTASVVCLLNNFYPREAKVQWKVDNALQSGNSQESVTEQDSKDSTYSLSSTLTLSKADYEKHKLYACEVTHQGLSSPVTKSFNRGEC

SEQ ID NO: 20 - protein, 119 aa, Homo sapiens
YTFGQGTKLEIKRTVAAPSVFIFPPSDEQLKSGTASVVCLLNNFYPREAKVQWKVDNALQSGNSQESVTEQDSKDSTYSLSSTLTLSKADYEKHKLYACEVTHQGLSSPVTKSFNRGEC

SEQ ID NO: 21 - protein, 119 aa, Homo sapiens
FTFGPGTKVDIKRTVAAPSVFIFPPSDEQLKSGTASVVCLLNNFYPREAKVQWKVDNALQSGNSQESVTEQDSKDSTYSLSSTLTLSKADYEKHKLYACEVTHQGLSSPVTKSFNRGEC

SEQ ID NO: 22 - protein, 119 aa, Homo sapiens
LTFGGGTKVEIKRTVAAPSVFIFPPSDEQLKSGTASVVCLLNNFYPREAKVQWKVDNALQSGNSQESVTEQDSKDSTYSLSSTLTLSKADYEKHKLYACEVTHQGLSSPVTKSFNRGEC

SEQ ID NO: 23 - protein, 119 aa, Homo sapiens
ITFGQGTRLEIKRTVAAPSVFIFPPSDEQLKSGTASVVCLLNNFYPREAKVQWKVDNALQSGNSQESVTEQDSKDSTYSLSSTLTLSKADYEKHKLYACEVTHQGLSSPVTKSFNRGEC

SEQ ID NO: 24 - protein, 107 aa, Homo sapiens
RTVAAPSVFIFPPSDEQLKSGTASVVCLLNNFYPREAKVQWKVDNALQSGNSQESVTEQDSKDSTYSLSSTLTLSKADYEKHKLYACEVTHQGLSSPVTKSFNRGEC

SEQ ID NO: 25 - protein, 122 aa, Homo sapiens
MVLQTQVFISLLLWISGAYGDIVMTQSPDSLAVSLGERATINCKSSQSVLYSSNNKNYLAWYQQKPGQPPKLLIYWASTRESGVPDRFSGSGSGTDFTLTISSLQAEDVAVYYCQQYYSTPP

SEQ ID NO: 26 - protein, 6 aa, Artificial Sequence
  Description of Artificial Sequence Synthetic 6xHis tag
HHHHHH

SEQ ID NO: 27 - protein, 6 aa, Artificial Sequence
  Description of Artificial Sequence Synthetic 6xHis tag
HHHHHH

SEQ ID NO: 28 - protein, 145 aa, Homo sapiens
MSWAPVLLMLFVYCTGCGPQPVLHQPPAMSSALGTTIRLTCTLRNDHDIGVYSVYWYQQRPGHPPRFLLRYFSQSDKSQGPQVPPRFSGSKDVARNRGYLSISELQPEDEAMYYCAMGARSSEKEEREREWEEEMEPTAARTRVP

SEQ ID NO: 29 - protein, 142 aa, Mus sp.
MAWTSVLLMLLAHLTGCGPQPMVHQPPSASSSLGATIRLSCTLSNDHNIGIYSIYWYQQRPGHPPRFLLRYFSHSDKHQGPDIPPRFSGSKDTARNLGYLSISELQPEDEAVYYCAVGLRSHEKKRMEREWEGEKSYTDLGS

SEQ ID NO: 30 - protein, 171 aa, Mus sp.
MAWTSVLLMLLAHLTGKGTLGVQGFLAPPVALLCPSDGHASIFSGCGPQPMVHQPPSASSSLGATIRLSCTLSNDHNIGIYSIYWYQQRPGHPPRFLLRYFSHSDKHQGPDIPPRFSGSKDTARNLGYLSISELQPEDEAVYYCAVGLRSHEKKRMEREWEGEKSYTDLGS

SEQ ID NO: 31 - protein, 123 aa, Homo sapiens
MACRCLSFLLMGTFLSVSQTVLAQLDALLVFPGQVAQLSCTLSPQHVTIRDYGVSWYQQRAGSAPRYLLYYRSEEDHHRPADIPDRFSAAKDEAHNACVLTISPVQPEDDADYYCSVGYGFSP

SEQ ID NO: 32 - protein, 209 aa, Homo sapiens
MKLRVGQTLGTIPRQCEVLLLLLLLGLVDGVHHILSPSSAERSRAVGPGASVGSNRPSLWALPGRLLFQIIPRGAGPRCSPHRLPSKPQFWYVFGGGTQLTILGQPKSDPLVTLFLPSLKNLQPTRPHVVCLVSEFYPGTLVVDWKVDGVPVTQGVETTQPSKQTNNKYMVSSYLTLISDQWMPHSRYSCRVTHEGNTVEKSVSPAECS

SEQ ID NO: 33 - protein, 213 aa, Homo sapiens
MRPGTGQGGLEAPGEPGPNLRQRWPLLLLGLAVVTHGLLRPTAASQSRALGPGAPGGSSRSSLRSRWGRFLLQRGSWTGPRCWPRGFQSKHNSVTHVFGSGTQLTVLSQPKATPSVTLFPPSSEELQANKATLVCLMNDFYPGILTVTWKADGTPITQGVEMTTPSKQSNNKYAASSYLSLTPEQWRSRRSYSCQVMHEGSTVEKTVAPAECS

(19) United States
(12) Patent Application Publication
(10) Pub. No.: US 2010/0062950 A1
(43) Pub. Date: Mar. 11, 2010

(54) CONSTRUCTS AND LIBRARIES COMPRISING ANTIBODY SURROGATE KAPPA LIGHT CHAIN SEQUENCES

(76) Inventors: Ramesh R. Bhatt, Belmont, CA (US); Lawrence Horowitz, Atherton, CA (US)

Correspondence Address: Goodwin Procter LLP;Attn: Patent Administrator, 135 Commonwealth Drive, Menlo Park, CA, 94025-1105, US

(21) Appl. No.: 12/501,281

(22) Filed: Jul. 10, 2009

Related U.S. Application Data
(60) Provisional application No. 61/134,929, filed on Jul. 11, 2008.

(51) Int. Cl. C40B 40/02; C12P 21/08; C40B 40/10; C40B 40/06

(52) U.S. Cl. 506/14; 530/387.3; 506/18; 506/16

(57) ABSTRACT

The present invention concerns constructs and libraries comprising antibody surrogate κ light chain sequences. In particular, the invention concerns constructs comprising antibody surrogate κ light chain sequences, optionally partnered with another polypeptide, such as, for example, antibody heavy and/or light chain domain sequences, and libraries containing the same.

### CROSS-REFERENCE TO RELATED APPLICATIONS

This application claims priority under Section 119(e) and the benefit of U.S. Provisional Application Ser. No. 61/134,929 filed Jul. 11, 2008 the entire disclosures of which are incorporated herein by reference in their entirety.

### FIELD OF THE INVENTION

The present invention concerns constructs and libraries comprising antibody surrogate κ light chain sequences. In particular, the invention concerns constructs comprising antibody surrogate κ light chain sequences, optionally partnered with another polypeptide, such as, for example, antibody heavy and/or light chain domain sequences, and libraries containing the same.

### BACKGROUND OF THE INVENTION

Antibody (Ig) molecules produced by B-lymphocytes are built of heavy (H) and light (L) chains. The amino acid sequences of the amino terminal domains of the H and L chains are variable (V_{H} and V_{L}), especially at the three hypervariable regions (CDR1, CDR2, CDR3) that form the antigen combining site. The assembly of the H and L chains is stabilized by a disulfide bond between the constant region of the L chain (C_{L}) and the first constant region of the heavy chain (C_{H1}) and by non-covalent interactions between the V_{H} and V_{L} domains.

Various stages of B lymphocyte development are characterized by the rearrangement status of the Ig gene loci (see, e.g. Melchers, F. & Rolink, A., B-Lymphocyte Development and Biology, Paul, W. E., ed., 1999, Lippincott, Philadelphia). In humans and many animals, such as mice, the genes encoding the antibody H and L chains are assembled by stepwise somatic rearrangements of gene fragments encoding parts of the V regions in a defined ordered manner, where the μ heavy chain precedes the κ and λ, light chains (Burrows P D and Cooper M D, Curr Opin Immunol 9:239-44 (1997); Alt et al., Immunol Today 13:306-14 (1992); Bassing et al., Cell 109 Suppl.:S45-55 (2002)).

It is known that light chain (LC) rearrangement is initiated by signals that are driven through a pre-B-cell receptor (pre-BCR), formed by two μ heavy chains (μHCs) in conjunction with two covalently associated surrogate light chains (SLCs) composed of λ5 and VpreB molecules. Thus, precursors of B cells (pre-B cells) have been identified in the bone marrow as lymphocytes that produce μ heavy chains but instead of the fully developed light chains express a set of B lineage-specific genes called VpreB(1-3) and λ5, respectively.

The main isoform of human VpreB1 (CAG30495) is a 145 aa-long polypeptide. It has an Ig V domain-like structure, but lacks the last β-strand (β7) of a typical V domain, and has a carboxyl terminal end that shows no sequence homologies to any other proteins. VpreB2 has several isoforms, including a 142-amino acid mouse VpreB2 polypeptide (P13373), and a 171 amino acids long splice variant of the mouse VpreB2 sequence (CAA019641). VpreB1 and VpreB2 sequences have been disclosed in EP 0 269 127 and U.S. Pat. No. 5,182,205; Collins et al., Genome Biol. 5(10):R84 (2004); and Hollins et al., Proc. Natl. Acad. Sci. USA 86(14):5552-5556 (1989). The main isoform of human VpreB3 is a 123 aa-long protein (CAG30496), disclosed in Collins et al., Genome Biol. 5(10):R84 (2004).

VpreB(1-3) are non-covalently associated with another protein, λ5. The human λ5 is a 209-amino acid polypeptide (CAA01962), that carries an Ig C domain-like structure with strong homologies to antibody light chains and, towards its amino terminal end, two functionally distinct regions, one of which shows strong homology to the β7 strand of the Vλ, domains. A human λ5-like protein has 213 amino acids (NP_{—}064455) and shows about 84% sequence identity to the antibody λ, light chain constant region.

For further details, see the following review papers: Karasuyama et al., Adv. Immunol. 63:1-41 (1996); Melchers et al., Immunology Today 14:60-68 (1993); and Melchers, Proc. Natl. Acad. Sci. USA 96:2571-2573 (1999).

The VpreB and λ5 polypeptides together form a non-covalently associated, Ig light chain-like structure, which is called the surrogate light chain or pseudo light chain. On the surface of early preB cells, the surrogate light chain is disulfide-linked to membrane-bound Ig μ heavy chain in association with a signal transducer CD79a/CD79b heterodimer to form a B cell receptor-like structure, the pre-B cell receptor (pre-BCR).

Interestingly, maturation of B cell progenitors, cell surface expression and signaling of μ heavy chain (λHC) were observed even in the absence of λ5 expression. Thus, it has been reported that surrogate light chain deficient mice and surrogate light chain knockout mice are still cable of producing antibodies, thereby suggesting an alternative path for B cell development (see, for example, Kitamura et al., Cell 69:823-31 (1992); Rolink et al., Eur J Immunol 23:1284-8 (1993); Schuh et al., J Immunol 171:3343-7 (2003); Martensson et al., Int Immunol 11:453-60 (1999); Mundt et al., J Exp Med 193:435-45 (1991); Shimizu et al., J Immunol 168:6286-93 (2002)).

A κ-like B cell receptor (κ-like BCR) has been identified, utilizing a κ-like surrogate light chain (κ-like SLC) (Frances et al., EMBO J. 13:5937-43 (1994); Thompson et al., Immunogenetics 48:305-11 (1998); Rangel et al., J Biol Chem 280:17807-14 (2005)).

Rangel et al., J Biol Chem 280(18):17807-17814 (2005) report the identification and molecular characterization of a V_{κ}-like protein that is the product of an unrearranged V_{κ} gene, which turned out to the be identical to the cDNA sequence previously reported by Thompson et al., Immunogenetics 48:305-311 (1998). Whereas, Frances et al., EMBO J. 13:5937-43 (1994) reported the identification and characterization of a rearranged germline JCκ that has the capacity to associate with μ heavy chains at the surface of B cell precursors, thereby providing an alternative to the λ5 pathway for B cell development.

It has been proposed that κ-like and λ-like pre-BCRs work in concert to promote light chain rearrangement and ensure the maturation of B cell progenitors. For a review, see McKeller and Martinez-Valdez Seminars in Immunology 18:4043 (2006).

### SUMMARY OF THE INVENTION

In one aspect, the invention concerns a κ-like surrogate light chain (SLC) construct comprising a Vκ-like and/or a JCκ sequence.

In various embodiments, the κ-like SLC construct comprises a Vκ-like sequence, or a JCκ sequence, or both a Vκ-like sequence and a JCκ sequence.

In all embodiments, the κ-like SLC construct may be capable of specifically binding to a target.

In various additional embodiments, the in the κ-like SLC construct the Vκ-like sequence comprises SEQ ID NO: 2, without or without a signal sequence and with or without a C-terminal tail, or a fragment thereof, or the N-terminal signal peptide (amino acids 1-20) of SEQ ID NO: 2, and may additionally comprise at least part of the C-terminal tail from within SEQ ID NO: 2.

In a particular embodiment, the Vκ-like sequence is selected from the group comprising SEQ ID NOs: 7-18, with or without a signal sequence and with or without a C-terminal tail, or a fragment thereof.

In other embodiment, in the κ-like SLC constructs herein, the JCκ sequence comprises SEQ ID NO: 4, with or without an N-terminal extension, or a fragment thereof, or a sequence selected from the group consisting of SEQ ID NOs: 19-23, with or without an N-terminal extensions, or a fragment thereof.

In all embodiments, the κ-like SLC construct may be associated with an antibody heavy chain sequence.

In other embodiments, in the κ-like SLC construct the Vκ-like sequence comprises a C-terminal tail.

In still further embodiments, in the κ-like SLC constructs the JCκ sequence comprises an N-terminal extension.

In yet another embodiment, in the κ-like SLC constructs herein the Vκ-like sequence comprises a C-terminal tail and the JCκ sequence comprises an N-terminal extension.

In a different embodiment, the Vκ-like sequence is devoid of a C-terminal tail and the JCκ sequence is devoid of an N-terminal extension.

In all embodiments, if the construct is associated with or is connected to an antibody heavy chain sequence, the latter may be a full-length antibody heavy chain or a fragment thereof.

In one embodiment, in the κ-like SLC construct the Vκ-like sequence and the JCκ sequence are covalently linked to each other, including, without limitation, direct fusions, and linkage through a heterogenous linker, which may, for example, comprise a sequence of a native polypeptide or a fragment thereof, such a sequence of a therapeutic polypeptide or a fragment thereof.

In another embodiment, the heterogeneous linker comprises an antibody sequence, which may include antibody heavy and/or light chain variable and/or constant region sequences.

In a particular embodiment, the antibody light chain and heavy chain sequences, when present, are capable of binding an antigen, which may be the same as, or different from, the target to which said construct binds.

Thus, for example, the constructs herein may be bifunctional, trifunctional or, in general, multifunctional.

In other embodiments, the Vκ-like sequence comprises a C-terminal tail and the JCκ sequence comprises an N-terminal extension, one or both of which may be linked to a heterogeneous molecule, such as, for example, a peptide or a polypeptide.

In all embodiments, the κ-like SLC constructs may have improved pharmacokinetic profiles and/or potency, and/or other improved functional properties relative to an antibody with the same qualitative binding specicity.

In another aspect, the invention concerns a library comprising a collection of the κ-like SLC constructs herein

The library may be in the form of a display, such as a phage display, bacterial display, yeast display, ribosome display, mRNA display, DNA display, display on mammalian cells, spore display, viral display, display based on protein-DNA linkage, and microbead display.

In addition, the library may contain a collection of antibody sequences, such as antibody heavy and/or light chain sequences.

In other embodiments, the library comprises a collection of Vκ-like sequences, wherein the collection of Vκ-like sequences may comprise Vκ-like sequence variants that differ in their CDR sequences and/or in the C-terminal sequences.

In further embodiments, the library may comprise a collection of JCκ sequences, which may comprise JCκ sequence variants that differ in their N-terminal extensions.

In all embodiments, when an antibody heavy chain comprising variable region sequences is present, the polypeptide of the present invention and the antibody heavy chain variable region sequences may bind to the same or to different targets.

### BRIEF DESCRIPTION OF THE DRAWINGS (SEQ IDS AT END OF DOCUMENT)

FIG. 1: CDR analogous regions and the C-terminal tail of the VpreB1 domain span a considerable distance. Light grey: CDR residues; dark grey: framework residues.

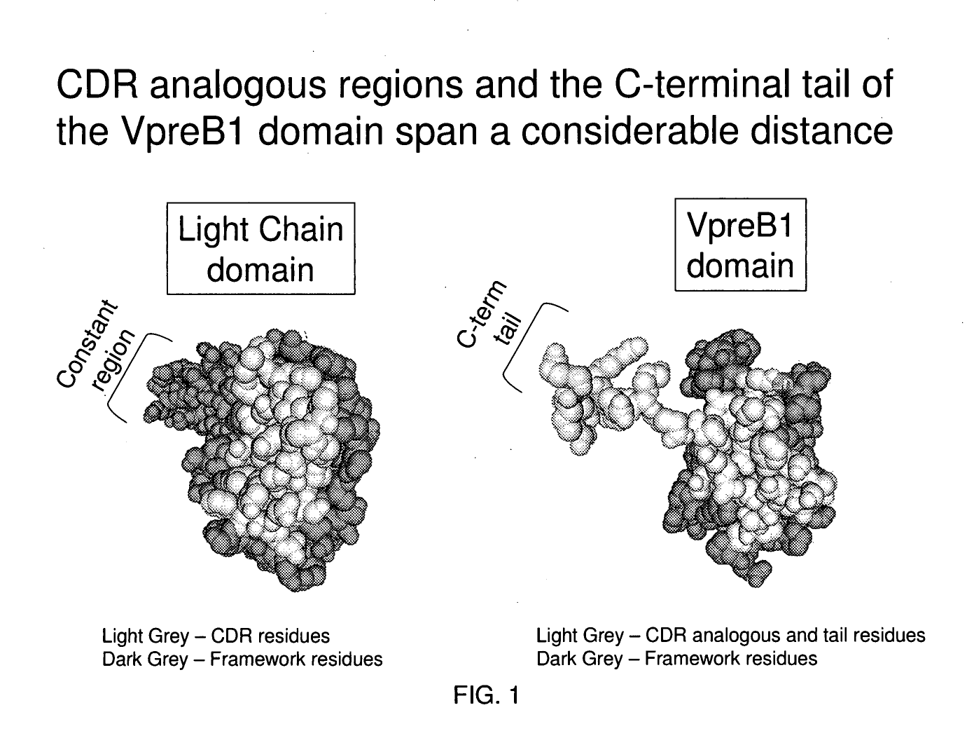

FIG. 2 shows the nucleotide sequence of a human Vκ-like nucleic acid (SEQ ID NO: 1) and the amino acid sequence of the encoded protein (SEQ ID NO: 2).

FIG. 3 shows the nucleotide sequence of a human JCκ nucleic acid (SEQ ID NO: 3), and the amino acid sequence of the encoded protein (SEQ ID NO: 4).

FIG. 4 shows the alignment of κ-like surrogate light chains (AJ004956 Vκ-like, SEQ ID NO: 2 and AAB32987 human JCκ, SEQ ID NO: 4) with human Ig variable and constant κ light chains (VκIV_B3, SEQ ID NO: 5; Constant kappa, SEQ ID NO: 6). Vκ-like is an unrearranged VκIV gene family member, but has a unique C-terminal extension; JCκ shares identity to kappa J and constant regions, but has a unique N-terminus; CDR1 and CDR2 are conserved, but CDR3 is interrupted.

FIG. 5 is a schematic illustration of various heterodimeric surrogate κ light chain deletion variants. In the “full length” construct, both the Vκ-like and JCκ sequence retains the C- and N-terminal extensions (tails), respectively. In the dJ variant, the N-terminal extension of JCκ has been deleted. In the dVκ tail variants, the C-terminal extension of the Vκ-like sequence had been removed but the N-terminal extension of JCκ is retained. In the “short kappa” variant, both the C-terminal tail of the Vκ-like sequence and the N-terminal extension of the JCκ sequence are retained.

FIG. 6: κ-like light chain deletion and single chain constructs, which can be used individually or with another protein, such as an antibody heavy chain or a fragment thereof.

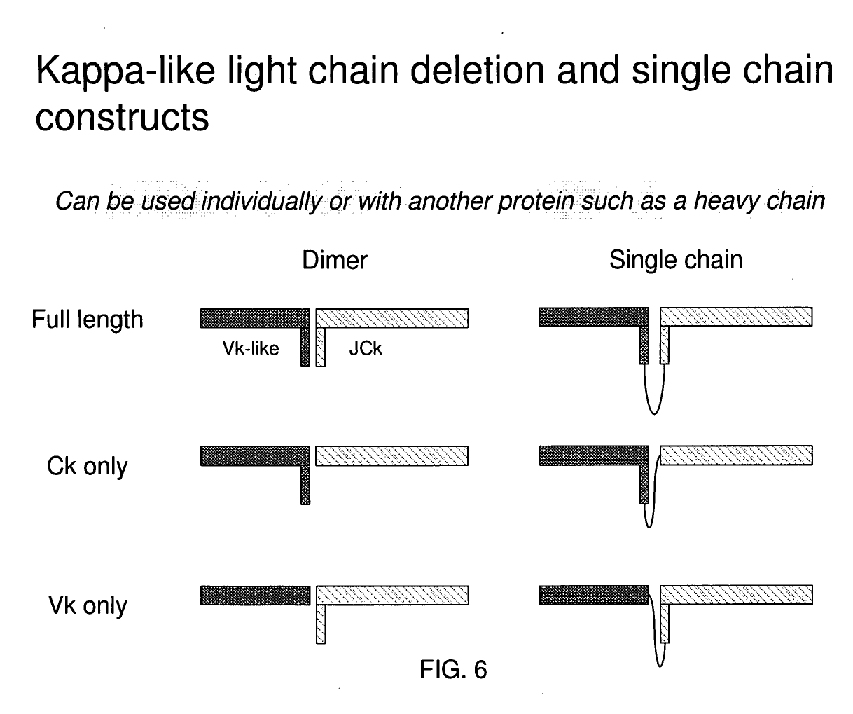

FIG. 7: Incorporating combinatorial functional diversity into κ-like surrogate light chain constructs. Red lines indicate appended diversity, such as a peptide library.

FIG. 8: Light chains are products of gene rearrangement and RNA processing.

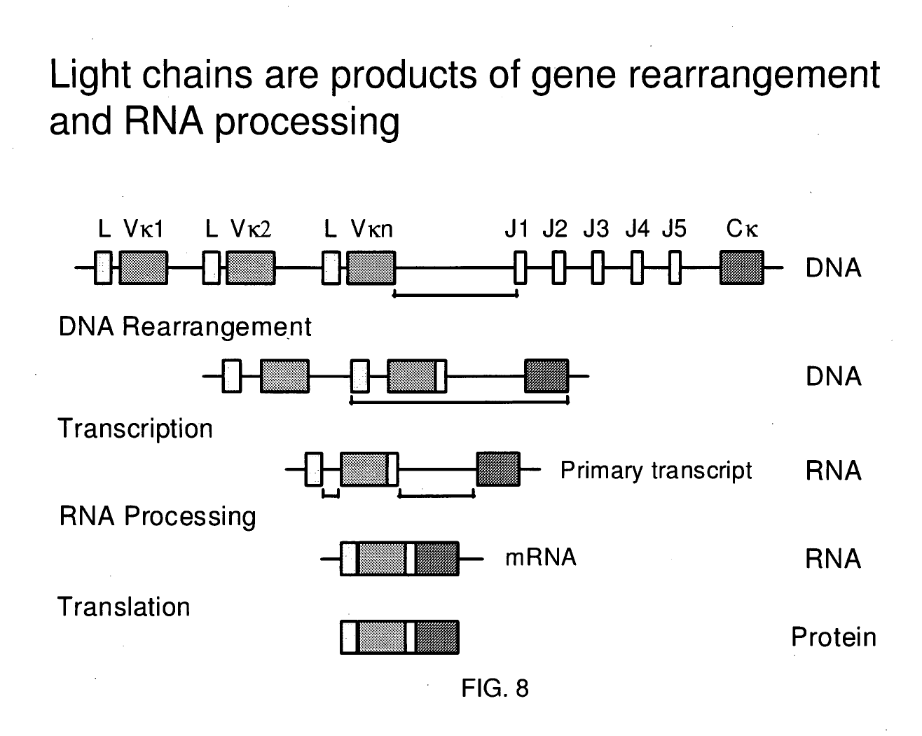

FIG. 9 illustrates that Vκ-like protein is derived from unrearranged VκIV-gene transcription and translation. VκIV is one of seventy-one VL germiline genes. Since there are an additional 70 VL germline genes capable of creating Vκ-like proteins, there are 39 more κV genes and 31 more λV genes.

FIG. 10: Predicted amino acid sequences of Vκ-like proteins possible from all Vκ families, each bearing different lengths of extensions (SEQ ID NOs: 7-18) aligned with AJ004956 Vκ-like prototype sequence (SEQ ID NO: 2).

FIG. 11: JCκ is a product of processed RNA from unrearranged J and C germlines JCκ is one of forty-five JC germline combinations. There are an additional 44 VL germline genes capable of creating JCκ-like proteins 4 more Jκ genes to combine with Cκ and 4 Jλ, genes to combine with 10 Cλ genes (40 total).

FIG. 12: Predicted JCκ-like from remaining kappa J-constant region rearrangements (J1-J5Cκ) (SEQ ID NOs: 19-23).

FIG. 13: Schematic illustrating of adding functionality to κ-like surrogate light chain components. Bifunctional and trifunctional structures are illustrated. A: scFv constrained fusion; B: Vκ-like scFv fusion; C: JCκ scFv fusion; D: SLC dual fusion.

FIG. 14: Illustrative types of surrogate light chain functional tail extensions.

FIG. 15: Illustrative surrogate light chain GLP-1 fusions.

FIG. 16: Illustrative κ-like and λ-like surrogate light chain functional chimeras.

FIG. 17: amino acid sequences of human VpreB1 (SEQ ID NO: 30), mouse VpreB2 (SEQ ID NOS:31), human VpreB3 (SEQ ID NO: 33), human λ5 sequence (SEQ ID NO: 34), and human λ5-like sequence (SEQ ID NO: 35).

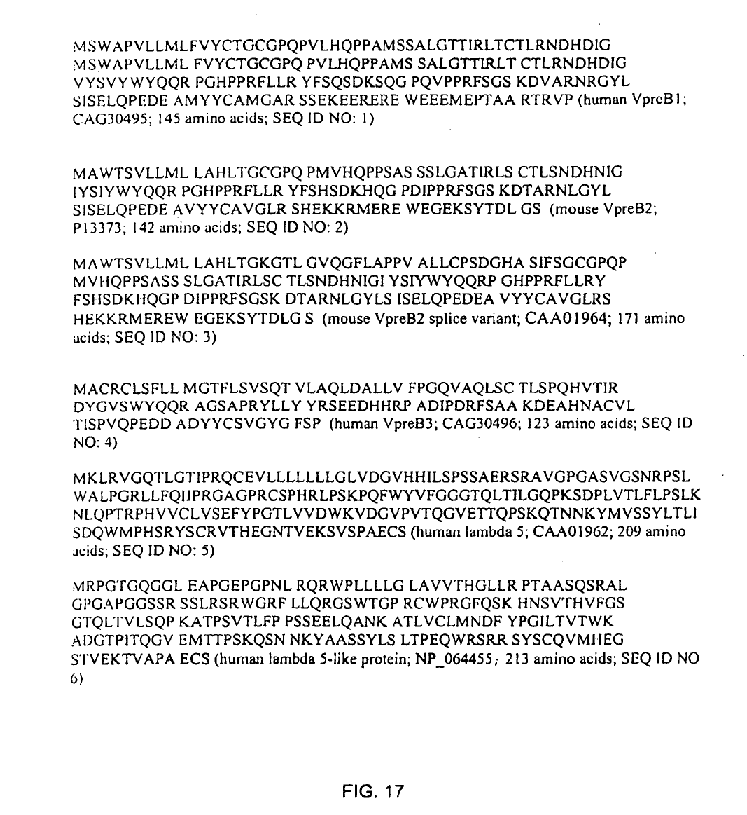

### DETAILED DESCRIPTION OF THE INVENTION

### A. Definitions

Unless defined otherwise, technical and scientific terms used herein have the same meaning as commonly understood by one of ordinary skill in the art to which this invention belongs. Singleton et al., Dictionary of Microbiology and Molecular Biology 2nd ed., J. Wiley & Sons (New York, N.Y. 1994), provides one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described. For purposes of the present invention, the following terms are defined below.

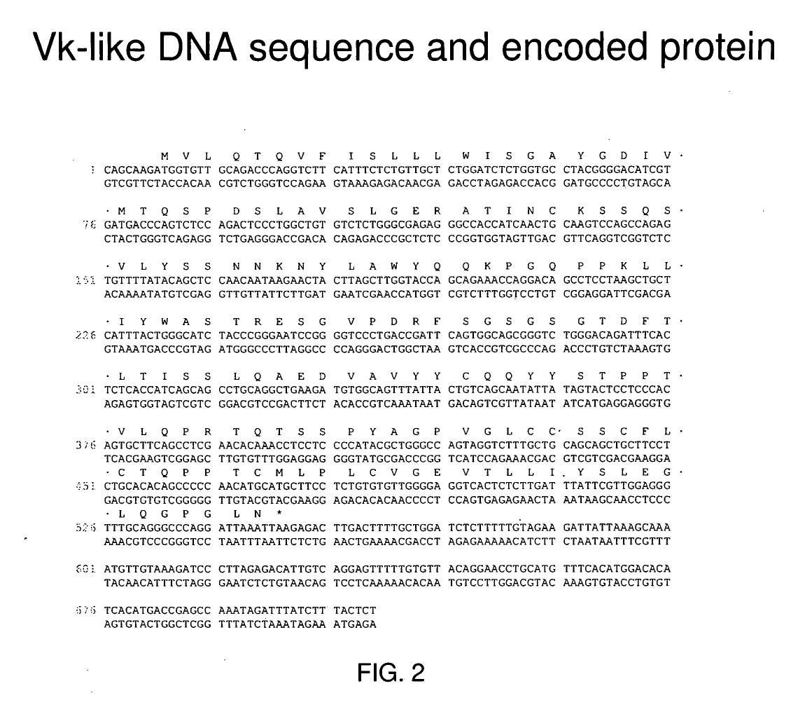

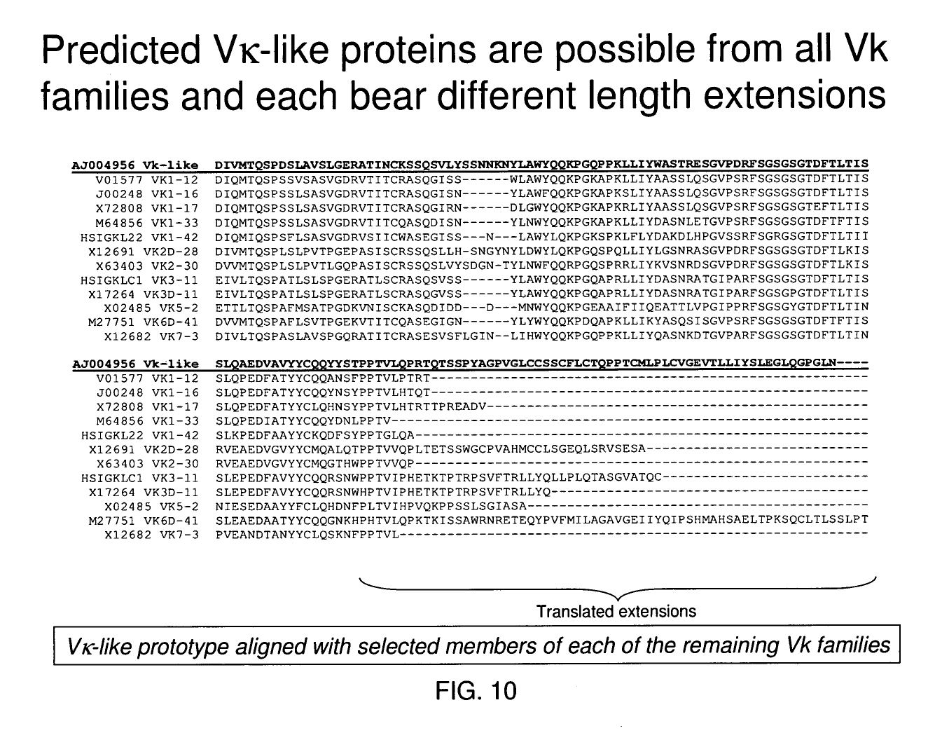

The terms “κ-like surrogate light chain variable domain,” “Vκ-like SLC,” and “Vκ-like” are used interchangeably, and refer to any native sequence polypeptide that is the product of an unrearranged 17 h gene, and variants thereof. Native sequence “Vκ-like” polypeptides specifically include, without limitation, the human κ-like polypeptide AJ004956 shown in FIG. 2 (SEQ ID NO: 2); and the human Vκ-like polypeptides shown in FIG. 10 (SEQ ID NOs: 7-18), as well as homologs in non-human mammalian species, in particular species which, like humans, generate antibody diversity predominantly by gene rearrangement and/or hypermutation, such as rodents, e.g. mice and rats, and non-human higher primates. In one embodiment, variants of native sequence Vκ-like polypeptides comprise a C-terminal extension (tail) relative to antibody κ light chain sequences. In a particular embodiment, variants of native sequence Vκ-like polypeptides retain at least part, and preferably all, of the unique C-terminal extension (tail) that distinguishes the Vκ-like polypeptides from the corresponding antibody κ light chains. In another embodiment, the C-terminal tail of the variant Vκ-like polypeptide is a sequence not naturally associated with the rest of the sequence. In the latter embodiment, the difference between the C-terminal tail naturally present in the native Vκ-like sequence and the variant sequence may result from one or more amino acid alterations (substitutions, insertions, deletions, and/or additions), or the C-terminal tail may be identical with a tail present in nature in a different Vκ-like protein. Thus, for example, in any of the Vκ-like proteins listed in FIG. 10 (SEQ ID NOs: 2 and 7-18), the C-terminal extension (referred to as “translated extensions” in FIG. 10) may be replaced by the C-terminal extension of another Vκ-like protein and/or altered so that it differs from any naturally occurring C-terminal extension sequence. Alternatively or in addition, variants of native sequence Vκ-like polypeptides may contain one or more amino acid alterations in the part of the sequence that is identical to a native antibody κ variable domain sequence, in particular in one or more of the complementarity determining regions (CDRs) and/or framework residues of such sequence. Thus, the Vκ-like polypeptides may contain amino acid alterations in regions corresponding to one or more of antibody κ light chain CDR1, CDR2 and CDR3 sequences. In all instances, the variants can, and preferably do, include a C-terminal extension of at least four, or at least five, or at least six, or at least seven, or at least eight, or at least nine, or at least ten amino acids, preferably 4-100, or 4-90, or 4-80, or 4-70, or 4-60, or 4-50, or 4-45, or 4-40, or 4-35, or 4-30, or 4-25, or 4-20, or 4-15, or 4-10 amino acid residues relative to a native antibody κ light chain variable region sequence. As defined herein, Vκ-like polypeptide variant will be different from a native antibody κ or λ, light chain sequence or a fragment thereof, and will preferably retain at least about 65%, or at least about 70%, or at least about 75%, or at least about 80%, or at least about 85%, or at least about 90%, or at least about 95%, or at least about 98% sequence identity with a native sequence Vκ polypeptide. In another preferred embodiment, the Vκ-like polypeptide variant will be less then 95%, or less than 90%, or less then 85%, ore less than 80%, or less than 75%, or less then 70%, or less than 65%, or less than 60%, or less then 55%, or less than 50%, or less than 45%, or less than 40% identical in its amino acid sequence to a native antibody λ or κ light chain sequence. In other embodiments, the sequence identity is between about 40% and about 95%, or between about 45% and about 90%, or between about 50% and about 85%, or between about 55% and about 80%, or between about 60% and about 75%, or between about 60% and about 80%, or between about 65% and about 85%, or between about 65% and about 90%, or between about 65% and about 95%. In all embodiments, preferably the Vκ-like polypeptides are capable of binding to a target.

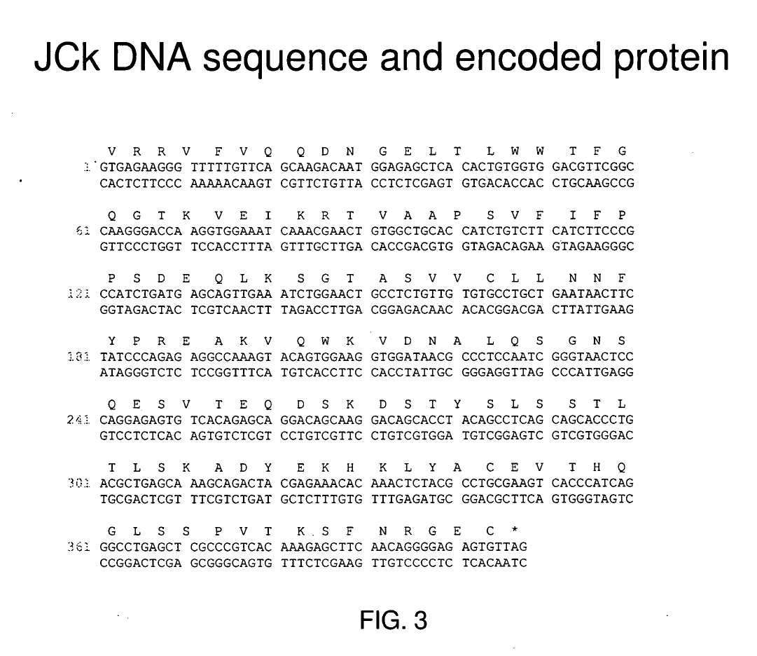

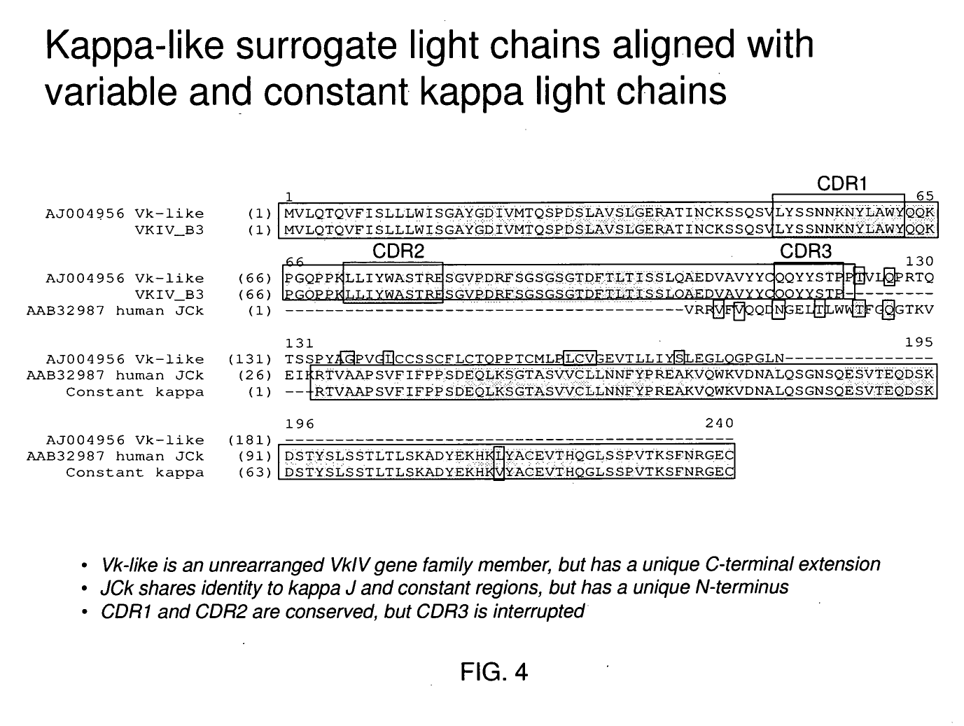

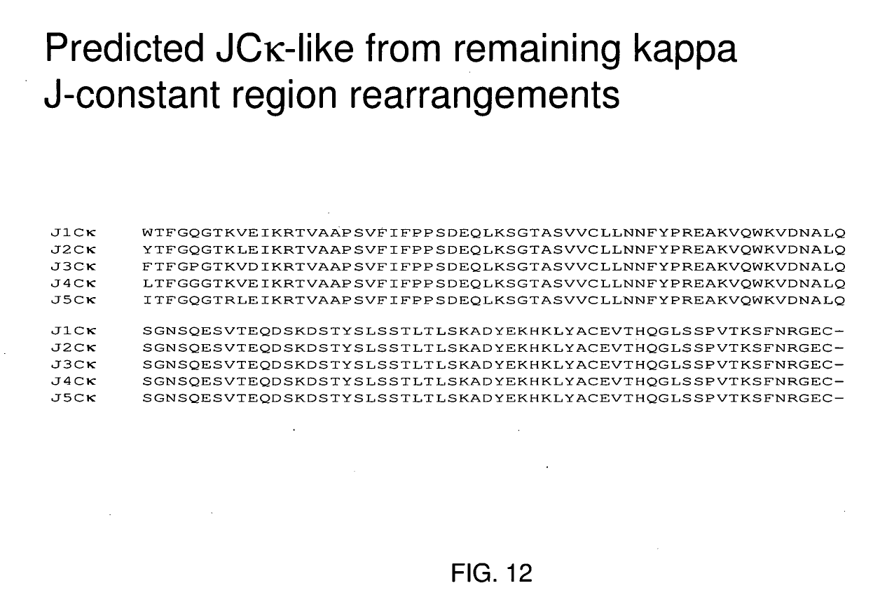

The terms “JCκ” and “JCκ-like” are used interchangeably, and refer to native sequence polypeptides that include a portion identical to a native sequence κJ-constant (C) region segment and a unique N-terminal extension (tail), and variants thereof. Native sequence JCκ-like polypeptides include, without limitation, the AAB32987 human JCκ polyepeptide shown in FIGS. 3 and 4 (SEQ ID NO: 4) and the JCκ-like polypeptides shown in FIG. 12 (SEQ ID NOs: 19-23), as well as homologs in non-human mammalian species, in particular species which, like humans, generate antibody diversity predominantly by gene rearrangement and/or hypermutation, such as rodents, e.g. mice and rats, and non-human higher primates. In one embodiment, variants of native sequence JCκ-like polypeptides comprise an N-terminal extension (tail) that distinguishes them from an antibody JC segment. In a particular embodiment, variants of native sequence JCκ-like polypeptides retain at least part, and preferably all, of the unique N-terminal extension (tail) that distinguishes the JCκ-like polypeptides from the corresponding antibody κ light chain JC segments. In another embodiment, the N-terminal tail of the variant JCκ-like polypeptide is a sequence not naturally associated with the rest of the sequence. In the latter embodiment, the difference between the N-terminal tail naturally present in the native JCκ-like sequence and the variant sequence may result from one or more amino acid alterations (substitutions, insertions, deletions, and/or additions), or the N-terminal tail may be identical with a tail present in nature in a different JCκ-like protein. Thus, for example, in any of the JCκ-like proteins listed in FIG. 12, the N-terminal extension may be replaced by the N-terminal extension of another JCκ-like protein and/or altered so that it differs from any naturally occurring N-terminal extension sequence. Alternatively or in addition, variants of native sequence JCκ-like polypeptides may contain one or more amino acid alterations in the part of the sequence that is identical to a native antibody κ variable domain JC sequence. In all instances, the variants can, and preferably do, include an N-terminal extension (unique N-terminus) of at least four, or at least five, or at least six, or at least seven, or at least eight, or at least nine, or at least ten amino acids, preferably 4-100, or 4-90, or 4-80, or 4-70, or 4-60, 4-50, or 4-45, or 4-40, or 4-35, or 4-30, or 4-25, or 4-20, or 4-15, or 4-10 amino acid residues relative to a native antibody κ light chain JC sequence. The JCκ-like polypeptide variant, as defined herein, will be different from a native antibody λ or κ light chain JC sequence, or a fragment thereof, and will preferably retain at least about 65%, or at least about 70%, or at least about 75%, or at least about 80%, or at least about 85%, or at least about 90%, or at least about 95%, or at least about 98% sequence identity with a native sequence JC polypeptide. In another preferred embodiment, the JCκ-like polypeptide variant will be less then 95%, or less than 90%, or less then 85%, ore less than 80%, or less than 75%, or less then 70%, or less than 65%, or less than 60% identical in its amino acid sequence to a native antibody λ or κ light chain JC sequence. In other embodiments, the sequence identity is between about 40% and about 95%, or between about 45% and about 90%, or between about 50% and about 85%, or between about 55% and about 80%, or between about 60% and about 75%, or between about 60% and about 80%, or between about 65% and about 85%, or between about 65% and about 90%, or between about 65% and about 95%.

Percent amino acid sequence identity may be determined using the sequence comparison program NCBI-BLAST2 (Altschul et al., Nucleic Acids Res. 25:3389-3402 (1997)). The NCBI-BLAST2 sequence comparison program may be downloaded from http://www.ncbi.nlm.nih.gov or otherwise obtained from the National Institute of Health, Bethesda, Md. NCBI-BLAST2 uses several search parameters, wherein all of those search parameters are set to default values including, for example, unmask=yes, strand=all, expected occurrences=10, minimum low complexity length=15/5, multi-pass e-value=0.01, constant for multi-pass=25, dropoff for final gapped alignment=25 and scoring matrix=BLOSUM62.

The “κ-like” surrogate light chain sequence may be optionally conjugated to a heterogeneous amino acid sequence, or any other heterogeneous component, to form a “κ-like surrogate light chain construct” herein. Thus, the term, “κ-like surrogate light chain construct” is used in the broadest sense and includes any and all additional heterogeneous components, including a heterogeneous amino acid sequence, nucleic acid, and other molecules conjugated to a κ-like surrogate light chain sequence, wherein “conjugation” is defined below. In a preferred embodiment, the “κ-like surrogate light chain sequence” is capable of binding to a target. In a preferred embodiment, the “κ-like” surrogate light chain sequence is non-covalently or covalently associated with a JCκ-like sequence and/or an antibody heavy chain sequence or a fragment thereof. Covalent association includes direct fusions but also connection through a linker. Thus, for example, the Vκ-like and JCκ-like sequences may be connected via antibody light and/or heavy chain variable region sequences.

The term “VpreB” is used herein in the broadest sense and refers to any native sequence or variant VpreB polypeptide, specifically including, without limitation, human VpreB1 of SEQ ID NO: 30, mouse VpreB2 of SEQ ID NOS:31 and 32, human VpreB3 of SEQ ID NO: 33 and isoforms, including splice variants and variants formed by posttranslational modifications, other mammalian homologues thereof, especially in mammals which, like humans, generate antibody diversity primarily by gene rearrangement and/or hypermutation, such as rodents, e.g. mice and rats, as well as variants of such native sequence polypeptides.

The term “λ5” is used herein in the broadest sense and refers to any native sequence or variant λ5 polypeptide, specifically including, without limitation, human λ5 of SEQ ID NO: 34, human λ5-like protein of SEQ ID NO: 35, and their isoforms, including splice variants and variants formed by posttranslational modifications, other mammalian homologous thereof, especially in mammals which, like humans, generate antibody diversity primarily by gene rearrangement and/or hypermutation, such as rodents, e.g. mice and rats, as well a variants of such native sequence polypeptides.

In the context of the polypeptides of the present invention, the term “heterogeneous amino acid sequence,” relative to a first amino acid sequence, is used to refer to an amino acid sequence not naturally associated with the first amino acid sequence, at least not in the form it is present in the κ-like surrogate light chain constructs herein. Thus, a “heterogenous amino acid sequence” relative to a Vκ-like polypeptide is any amino acid sequence not associated with native Vκ-like polypeptide in its native environment, including, without limitation, JCκ sequences that are different from those JCκ sequences that, together with Vκ-like sequence, form a κ-like surrogate light chain, such as amino acid sequence variants, e.g. truncated and/or derivatized sequences. A “heterogeneous amino acid sequence” relative to a Vκ-like polypeptide also includes JCκ sequences covalently associated with, e.g. fused to, the Vκ-like polypeptide including native sequence JCκ, since in their native environment, the VκIV and JCκ sequences are not covalently associated, e.g. fused, to each other. In addition, a “heterogenous amino acid sequence” relative to a JCκ sequence can be any Vκ-like polypeptide sequence with which the JCκ sequence is not associated in their native environment. Further representative “heterogeneous amino acid sequences” relative to both Vκ-like and JCκ sequences include native and variant VpreB and λ5 sequences, and antibody light and heavy chain variable and constant region sequences. Generally speaking, the present invention provides heterogeneous amino acid sequences that are different from classic light chain amino acid sequences. For example, the heterogeneous amino acid sequences do not comprise the V-J joining of a classic light chain.

The terms “conjugate,” “conjugated,” and “conjugation” refer to any and all forms of covalent or non-covalent linkage, and include, without limitation, direct genetic or chemical fusion, coupling through a linker or a cross-linking agent, and non-covalent association, for example through Van der Waals forces, or by using a leucine zipper.

The term “fusion” is used herein to refer to the combination of amino acid sequences of different origin in one polypeptide chain by in-frame combination of their coding nucleotide sequences. The term “fusion” explicitly encompasses internal fusions, i.e., insertion of sequences of different origin within a polypeptide chain, in addition to fusion to one of its termini

As used herein, the term “target” is a substance that interacts with a polypeptide herein. Targets, as defined herein, specifically include antigens with which the VκIV- or JCκ-containing constructs of the present invention interact. Preferably, interaction takes place by direct binding.

As used herein, the terms “peptide,” “polypeptide” and “protein” all refer to a primary sequence of amino acids that are joined by covalent “peptide linkages.” In general, a peptide consists of a few amino acids, typically from about 2 to about 50 amino acids, and is shorter than a protein. The term “polypeptide,” as defined herein, encompasses peptides and proteins.

The term “amino acid” or “amino acid residue” typically refers to an amino acid having its art recognized definition such as an amino acid selected from the group consisting of: alanine (Ala); arginine (Arg); asparagine (Asn); aspartic acid (Asp); cysteine (Cys); glutamine (Gln); glutamic acid (Glu); glycine (Gly); histidine (His); isoleucine (Ile): leucine (Leu); lysine (Lys); methionine (Met); phenylalanine (Phe); proline (Pro); serine (Ser); threonine (Thr); tryptophan (Trp); tyrosine (Tyr); and valine (Val) although modified, synthetic, or rare amino acids may be used as desired. Thus, modified and unusual amino acids listed in 37 CFR 1.822(b)(4) are specifically included within this definition and expressly incorporated herein by reference Amino acids can be subdivided into various sub-groups. Thus, amino acids can be grouped as having a nonpolar side chain (e.g., Ala, Cys, Ile, Leu, Met, Phe, Pro, Val); a negatively charged side chain (e.g., Asp, Glu); a positively charged side chain (e.g., Arg, His, Lys); or an uncharged polar side chain (e.g., Asn, Cys, Gln, Gly, His, Met, Phe, Ser, Thr, Trp, and Tyr) Amino acids can also be grouped as small amino acids (Gly, Ala), nucleophilic amino acids (Ser, His, Thr, Cys), hydrophobic amino acids (Val, Leu, Ile, Met, Pro), aromatic amino acids (Phe, Tyr, Trp, Asp, Glu), amides (Asp, Glu), and basic amino acids (Lys, Arg).

The term “polynucleotide(s)” refers to nucleic acids such as DNA molecules and RNA molecules and analogues thereof (e.g., DNA or RNA generated using nucleotide analogues or using nucleic acid chemistry). As desired, the polynucleotides may be made synthetically, e.g., using art-recognized nucleic acid chemistry or enzymatically using, e.g., a polymerase, and, if desired, be modified. Typical modifications include methylation, biotinylation, and other art-known modifications. In addition, the nucleic acid molecule can be single-stranded or double-stranded and, where desired, linked to a detectable moiety.

The term “variant” with respect to a reference polypeptide refers to a polypeptide that possesses at least one amino acid mutation or modification (i.e., alteration) as compared to a native polypeptide. Variants generated by “amino acid modifications” can be produced, for example, by substituting, deleting, inserting and/or chemically modifying at least one amino acid in the native amino acid sequence.

An “amino acid modification” refers to a change in the amino acid sequence of a predetermined amino acid sequence. Exemplary modifications include an amino acid substitution, insertion and/or deletion.

An “amino acid modification at a specified position,” refers to the substitution or deletion of the specified residue, or the insertion of at least one amino acid residue adjacent the specified residue. By insertion “adjacent” a specified residue is meant insertion within one to two residues thereof. The insertion may be N-terminal or C-terminal to the specified residue.

An “amino acid substitution” refers to the replacement of at least one existing amino acid residue in a predetermined amino acid sequence with another different “replacement” amino acid residue. The replacement residue or residues may be “naturally occurring amino acid residues” (i.e. encoded by the genetic code) and selected from the group consisting of: alanine (Ala); arginine (Arg); asparagine (Asn); aspartic acid (Asp); cysteine (Cys); glutamine (Gln); glutamic acid (Glu); glycine (Gly); histidine (His); isoleucine (Ile): leucine (Leu); lysine (Lys); methionine (Met); phenylalanine (Phe); proline (Pro); serine (Ser); threonine (Thr); tryptophan (Trp); tyrosine (Tyr); and valine (Val). Substitution with one or more non-naturally occurring amino acid residues is also encompassed by the definition of an amino acid substitution herein.

A “non-naturally occurring amino acid residue” refers to a residue, other than those naturally occurring amino acid residues listed above, which is able to covalently bind adjacent amino acid residues(s) in a polypeptide chain. Examples of non-naturally occurring amino acid residues include norleucine, ornithine, norvaline, homoserine and other amino acid residue analogues such as those described in Ellman et al. Meth. Enzym. 202:301 336 (1991). To generate such non-naturally occurring amino acid residues, the procedures of Noren et al. Science 244:182 (1989) and Ellman et al., supra, can be used. Briefly, these procedures involve chemically activating a suppressor tRNA with a non-naturally occurring amino acid residue followed by in vitro transcription and translation of the RNA.

An “amino acid insertion” refers to the incorporation of at least one amino acid into a predetermined amino acid sequence. While the insertion will usually consist of the insertion of one or two amino acid residues, the present application contemplates larger “peptide insertions”, e.g. insertion of about three to about five or even up to about ten amino acid residues. The inserted residue(s) may be naturally occurring or non-naturally occurring as disclosed above.

An “amino acid deletion” refers to the removal of at least one amino acid residue from a predetermined amino acid sequence.

The term “mutagenesis” refers to, unless otherwise specified, any art recognized technique for altering a polynucleotide or polypeptide sequence. Preferred types of mutagenesis include error prone PCR mutagenesis, saturation mutagenesis, or other site directed mutagenesis.

“Site-directed mutagenesis” is a technique standard in the art, and is conducted using a synthetic oligonucleotide primer complementary to a single-stranded phage DNA to be mutagenized except for limited mismatching, representing the desired mutation. Briefly, the synthetic oligonucleotide is used as a primer to direct synthesis of a strand complementary to the single-stranded phage DNA, and the resulting double-stranded DNA is transformed into a phage-supporting host bacterium. Cultures of the transformed bacteria are plated in top agar, permitting plaque formation from single cells that harbor the phage. Theoretically, 50% of the new plaques will contain the phage having, as a single strand, the mutated form; 50% will have the original sequence. Plaques of interest are selected by hybridizing with kinased synthetic primer at a temperature that permits hybridization of an exact match, but at which the mismatches with the original strand are sufficient to prevent hybridization. Plaques that hybridize with the probe are then selected, sequenced and cultured, and the DNA is recovered.

In the context of the present invention, the term “antibody” (Ab) is used to refer to a native antibody from a classically recombined heavy chain derived from V(D)J gene recombination and a classically recombined light chain also derived from VJ gene recombination, or a fragment thereof.

A “native antibody” is heterotetrameric glycoprotein of about 150,000 daltons, composed of two identical light (L) chains and two identical heavy (H) chains. Each light chain is linked to a heavy chain by covalent disulfide bond(s), while the number of disulfide linkages varies between the heavy chains of different immunoglobulin isotypes. Each heavy and light chain also has regularly spaced intrachain disulfide bridges. Each heavy chain has, at one end, a variable domain (V_{H}) followed by a number of constant domains. Each light chain has a variable domain at one end (V_{L}) and a constant domain at its other end; the constant domain of the light chain is aligned with the first constant domain of the heavy chain, and the light chain variable domain is aligned with the variable domain of the heavy chain. Particular amino acid residues are believed to form an interface between the light- and heavy-chain variable domains, Chothia et al., J. Mol. Biol. 186:651 (1985); Novotny and Haber, Proc. Natl. Acad. Sci. U.S.A. 82:4592 (1985).

The term “variable” with reference to antibody chains is used to refer to portions of the antibody chains which differ extensively in sequence among antibodies and participate in the binding and specificity of each particular antibody for its particular antigen. Such variability is concentrated in three segments called hypervariable regions both in the light chain and the heavy chain variable domains. The more highly conserved portions of variable domains are called the framework region (FR). The variable domains of native heavy and light chains each comprise four FRs (FR1, FR2, FR3 and FR4, respectively), largely adopting a β-sheet configuration, connected by three hypervariable regions, which form loops connecting, and in some cases forming part of, the β-sheet structure. The hypervariable regions in each chain are held together in close proximity by the FRs and, with the hypervariable regions from the other chain, contribute to the formation of the antigen-binding site of antibodies (see Kabat et al., Sequences of Proteins of Immunological Interest, 5th Ed. Public Health Service, National Institutes of Health, Bethesda, Md. (1991), pages 647-669). The constant domains are not involved directly in binding an antibody to an antigen, but exhibit various effector functions, such as participation of the antibody in antibody-dependent cellular toxicity.

The term “hypervariable region” when used herein refers to the amino acid residues of an antibody which are responsible for antigen-binding. The hypervariable region comprises amino acid residues from a “complementarity determining region” or “CDR” (i.e., residues 30-36 (L1), 46-55 (L2) and 86-96 (L3) in the light chain variable domain and 30-35 (H1), 47-58 (H2) and 93-101 (H3) in the heavy chain variable domain; MacCallum et al., J Mol Biol. 262(5):732-45 (1996).

The term “framework region” refers to the art recognized portions of an antibody variable region that exist between the more divergent CDR regions. Such framework regions are typically referred to as frameworks 1 through 4 (FR1, FR2, FR3, and FR4) and provide a scaffold for holding, in three-dimensional space, the three CDRs found in a heavy or light chain antibody variable region, such that the CDRs can form an antigen-binding surface.

Depending on the amino acid sequence of the constant domain of their heavy chains, antibodies can be assigned to different classes. There are five major classes of antibodies IgA, IgD, IgE, IgG, and IgM, and several of these may be further divided into subclasses (isotypes), e.g., IgG1, IgG2, IgG3, IgG4, IgA, and IgA2.

The heavy-chain constant domains that correspond to the different classes of immunoglobulins are called α, δ, ε, γ, and μ, respectively.

The “light chains” of antibodies from any vertebrate species can be assigned to one of two clearly distinct types, called kappa (κ) and lambda (λ), based on the amino acid sequences of their constant domains. Any reference to an antibody light chain herein includes both κ and λ light chains.

“Antibody fragments” comprise a portion of a full length antibody, generally the antigen binding or a variable domain thereof. Examples of antibody fragments include, but are not limited to, Fab, Fab′, F(ab′)₂, scFv, (scFv)₂, dAb, and complementarity determining region (CDR) fragments, linear antibodies, single-chain antibody molecules, minibodies, diabodies, multispecific antibodies formed from antibody fragments, and, in general, polypeptides that contain at least a portion of an immunoglobulin that is sufficient to confer specific antigen binding to the polypeptide.

“Single-chain Fv” or “sFv” antibody fragments comprise the V_{H} and V_{L} domains of antibody, wherein these domains are present in a single polypeptide chain. Generally, the Fv polypeptide further comprises a polypeptide linker between the V_{H} and V_{L} domains which enables the sFv to form the desired structure for antigen binding. For a review of sFv see Plückthun in The Pharmacology of Monoclonal Antibodies, vol. 113, Rosenburg and Moore eds. Springer-Verlag, New York, pp. 269-315 (1994). Single-chain antibodies are disclosed, for example in WO 88/06630 and WO 92/01047.

Diabodies are bivalent, bispecific antibodies in which V_{H} and V_{L} domains are expressed on a single polypeptide chain, but using a linker that is too short to allow for pairing between the two domains on the same chain, thereby forcing the domains to pair with complementary domains of another chain and creating two antigen binding sites (see e.g., Holliger, P., et al., Proc. Natl. Acad. Sci. USA 90:6444 6448 (1993), and Poljak, R. J., et al., Structure 2:1121 1123 (1994)).

The term “minibody” is used to refer to an scFv-CH3 fusion protein that self-assembles into a bivalent dimer of 80 kDa (scFv-CH3)₂.

The term “aptamer” is used herein to refer to synthetic nucleic acid ligands that bind to protein targets with high specificity and affinity. Aptamers are known as potent inhibitors of protein function.

The term “affibody” is used to refer to engineered, target-specific, non-immunoglobulin binding proteins, which are typically based on the three-helix scaffold of the Z domain derived from staphylococcal protein A. The 58-amino acid Z domain is derived from one of five homologous domains (the B domain) in Staphylococcus aureus protein A (SPA). SPA binds strongly to the Fc region of immunoglobulins, and Z was originally developed as a stabilized gene fusion partner for affinity purification of recombinant proteins by using IgG-containing resins. The structure of a complex between the B domain of SPA and an Fc fragment shows that the binding surface consists of residues that are exposed on helices 1 and 2, whereas helix 3 is not directly involved in binding. Affibodies are usually selected from combinatorial libraries in which typically 13 residues at the Fc-binding surface of helices 1 and 2 are randomized. Specific binders to target proteins are then identified by biopanning the phage-displayed library against desired targets. Such affibodies can be used as an alternative to immunoglobulins in various biochemical assays and clinical applications.

A dAb fragment (Ward et al., Nature 341:544 546 (1989)) consists of a V_{H} domain or a VL domain.

As used herein the term “antibody binding regions” refers to one or more portions of an immunoglobulin or antibody variable region capable of binding an antigen(s). Typically, the antibody binding region is, for example, an antibody light chain (VL) (or variable region thereof), an antibody heavy chain (VH) (or variable region thereof), a heavy chain Fd region, a combined antibody light and heavy chain (or variable region thereof) such as a Fab, F(ab′)₂, single domain, or single chain antibody (scFv), or a full length antibody, for example, an IgG (e.g., an IgG1, IgG2, IgG3, or IgG4 subtype), IgA1, IgA2, IgD, IgE, or IgM antibody.

The term “epitope” as used herein, refers to a sequence of at least about 3 to 5, preferably at least about 5 to 10, or at least about 5 to 15 amino acids, and typically not more than about 500, or about 1,000 amino acids, which define a sequence that by itself, or as part of a larger sequence, binds to an antibody generated in response to such sequence. An epitope is not limited to a polypeptide having a sequence identical to the portion of the parent protein from which it is derived. Indeed, viral genomes are in a state of constant change and exhibit relatively high degrees of variability between isolates. Thus the term “epitope” encompasses sequences identical to the native sequence, as well as modifications, such as deletions, substitutions and/or insertions to the native sequence. Generally, such modifications are conservative in nature but non-conservative modifications are also contemplated. The term specifically includes “mimotopes,” i.e. sequences that do not identify a continuous linear native sequence or do not necessarily occur in a native protein, but functionally mimic an epitope on a native protein. The term “epitope” specifically includes linear and conformational epitopes.

The term “vector” is used to refer to a rDNA molecule capable of autonomous replication in a cell and to which a DNA segment, e.g., gene or polynucleotide, can be operatively linked so as to bring about replication of the attached segment. Vectors capable of directing the expression of genes encoding for one or more polypeptides are referred to herein as “expression vectors.” The term “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous Linking is accomplished by ligation at convenient restriction sites. If such sites do not exist, the synthetic oligonucleotide adaptors or linkers are used in accordance with conventional practice.

A “phage display library” is a protein expression library that expresses a collection of cloned protein sequences as fusions with a phage coat protein. Thus, the phrase “phage display library” refers herein to a collection of phage (e.g., filamentous phage) wherein the phage express an external (typically heterologous) protein. The external protein is free to interact with (bind to) other moieties with which the phage are contacted. Each phage displaying an external protein is a “member” of the phage display library.

The term “filamentous phage” refers to a viral particle capable of displaying a heterogenous polypeptide on its surface, and includes, without limitation, f1, fd, Pf1, and M13. The filamentous phage may contain a selectable marker such as tetracycline (e.g., “fd-tet”). Various filamentous phage display systems are well known to those of skill in the art (see, e.g., Zacher et al. Gene 9: 127-140 (1980), Smith et al. Science 228: 1315-1317 (1985); and Parmley and Smith Gene 73: 305-318 (1988)).

The term “panning” is used to refer to the multiple rounds of screening process in identification and isolation of phages carrying compounds, such as antibodies, with high affinity and specificity to a target.

The term “dominant negative” is used herein to refer to a polypeptide variant or polypeptide fragment acting as an antagonist of at least some biological properties of a parent or related protein.

### B. Detailed Description

Techniques for performing the methods of the present invention are well known in the art and described in standard laboratory textbooks, including, for example, Ausubel et al., Current Protocols of Molecular Biology, John Wiley and Sons (1997); Molecular Cloning: A Laboratory Manual, Third Edition, J. Sambrook and D. W. Russell, eds., Cold Spring Harbor, N.Y., USA, Cold Spring Harbor Laboratory Press, 2001; O'Brian et al., Analytical Chemistry of Bacillus Thuringiensis, Hickle and Fitch, eds., Am. Chem. Soc., 1990; Bacillus thuringiensis: biology, ecology and safety, T. R. Glare and M. O′Callaghan, eds., John Wiley, 2000; Antibody Phage Display, Methods and Protocols, Humana Press, 2001; and Antibodies, G. Subramanian, ed., Kluwer Academic, 2004. Mutagenesis can, for example, be performed using site-directed mutagenesis (Kunkel et al., Proc. Natl. Acad. Sci. USA 82:488-492 (1985)). PCR amplification methods are described in U.S. Pat. Nos. 4,683,192, 4,683,202, 4,800,159, and 4,965,188, and in several textbooks including “PCR Technology: Principles and Applications for DNA Amplification”, H. Erlich, ed., Stockton Press, New York (1989); and PCR Protocols: A Guide to Methods and Applications, Innis et al., eds., Academic Press, San Diego, Calif. (1990).

The present invention concerns constructs and libraries comprising antibody surrogate light chain sequences.

K-like Surrogate Light Chain Constructs

The Surrogate Light Chain (SLC) is a developmentally regulated polypeptide that naturally associates with the newly emerging heavy chains of the developing B cell receptor. Studies of heavy chain and surrogate light chains have shown in some instances that they can bind self antigens. It is well established that B cells containing surrogate light chain are well tolerated and can be found circulating under normal conditions. By extension therapeutic VH surrogate light chain heteromeric proteins may more readily bind self antigens and be well tolerated as therapeutic agents. An important distinction is that the surrogate light chain is not an antibody light chain. It is distinctly composed of two separate polypeptides that have not undergone classical light chain VJ rearrangement for their expression, yet still associate with a classical antibody heavy chain.

A well described lambda-like surrogate light chains exists, but there is also a body of evidence supporting the notion for additional SLCs, specifically a surrogate kappa-like light chain. This is supported in surrogate light chain knockout mice, which still are capable of producing antibodies, thereby suggesting an alternative path for B cell development. One candidate surrogate light is the κ-like light chain. The κ-like light chain is the germline VκIV gene partnered with a JCκ fusion gene. In each of these genes a peptidic extension exists in the vicinity surrounding a site analogous for CDR3. As these two proteins do not appear to recombine at the genomic level it is likely their association to a heavy chain are mutually exclusive of each other and analogous to the associations described for the λ-like surrogate light chain Importantly the peptide extensions seen in these genes provide opportunities to incorporate additional diversity or functionality.

As Vκ-like and the JCκ-like genes encode proteins that can function, independently from each other, as surrogate light chains, the κ-like surrogate light chain constructs of the present invention specifically include constructs comprising Vκ-like sequences without JCκ-like sequences, and JCκ-like sequences without Vκ-like sequences.

In one aspect, the present invention provides constructs comprising Vκ-like and/or JCκ sequences and having the ability to bind a target. The target can, for example, be any peptide or polypeptide that is a binding partner for the Vκ-like and/or JCκ sequence, or a construct containing such sequence(s). Targets specifically include all types of targets generally referred to as “antigens” in the context of antibody binding.

When the κ-like surrogate light chain constructs of the present invention comprise both a Vκ-like and a JCκ sequence, the two sequences are typically independent polypeptides, not fused to each other, but may also be associated non-covalently, or may be linked to each other by a covalent linker, such as a peptide linker and/or a linker comprising antibody sequences.

The constructs of the present invention include, without limitation, conjugates of VκIV and/or JCκ sequences to heterogeneous amino acid sequences. Binding to the heterogeneous amino acid sequence can be either covalent or non-covalent, and may occur directly or through a linker, including peptide linkers.

The free ends of the Vκ-like and/or JCκ sequences, including their variants and fragments, are available for incorporating an additional diversity into the library of such sequences. For instance, a random peptide library can be appended or substituted to one of these free ends and panned for specific binding to a particular target. By combining the surrogate light chain identified to have the desired binding specificity with a heavy chain or heavy chain fragment to the same target, a molecule can be created that has the ability to bind to the cognate target on two distinct places. This tandem binding, or “chelating” effect, strongly reinforces the binding to a single target, similarly to the avidity effects seen in dimeric immunoglobulins. It is also possible to use components binding to different targets. Thus, for example, the surrogate light chain component with the desired binding specificity can be combined with an antibody heavy chain or heavy fragment binding to a different target. For instance, the surrogate light chain component may bind a tumor antigen while the antibody heavy chain or heavy chain fragment may bind to effector cells. This way, a single entity with targeting and anti-tumor activity can be created. In a particular embodiment, the appendage or the polypeptide that connects the Vκ-like and/or JCκ sequences can be an antibody or antibody fragments, such as a Fab or a scFv fragment. The incorporation of an antibody sequence will not only create a “chelating” effect but can also generate bispecificity in a single molecule, without the need of a second independent arm, such as that found in bispecific antibodies. The two specificities may be to different parts of the same target, to disparate targets, or to a target antibody complex.

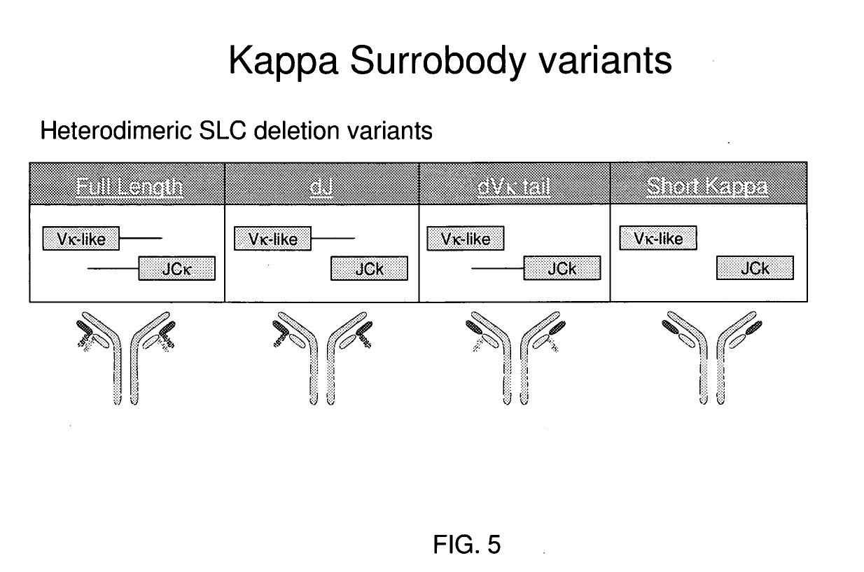

Specific examples of the polypeptide constructs herein include polypeptides in which a Vκ-like and/or JCκ sequence is associated with an antibody heavy chain, or a fragment thereof. Specific heterodimeric constructs, comprising both Vκ-like and JCκ sequences, are illustrated in FIG. 5. As shown in FIG. 5, in the κ-like surrogate light chain constructs of the present invention, the Vκ-like polypeptide and/or the JCκ polypeptide may contain the C- and N-terminal extensions, respectively, that are not present in similar antibody sequences. Alternatively, part or whole of the extension(s) can be removed from the κ-like surrogate light chain constructs herein.

Other κ-like surrogate light chain constructs, which can be used individually or can be further derivatized and/or associated with additional heterogeneous sequences, such as antibody heavy chain sequences, such as a full-length antibody heavy chain or a fragment thereof.

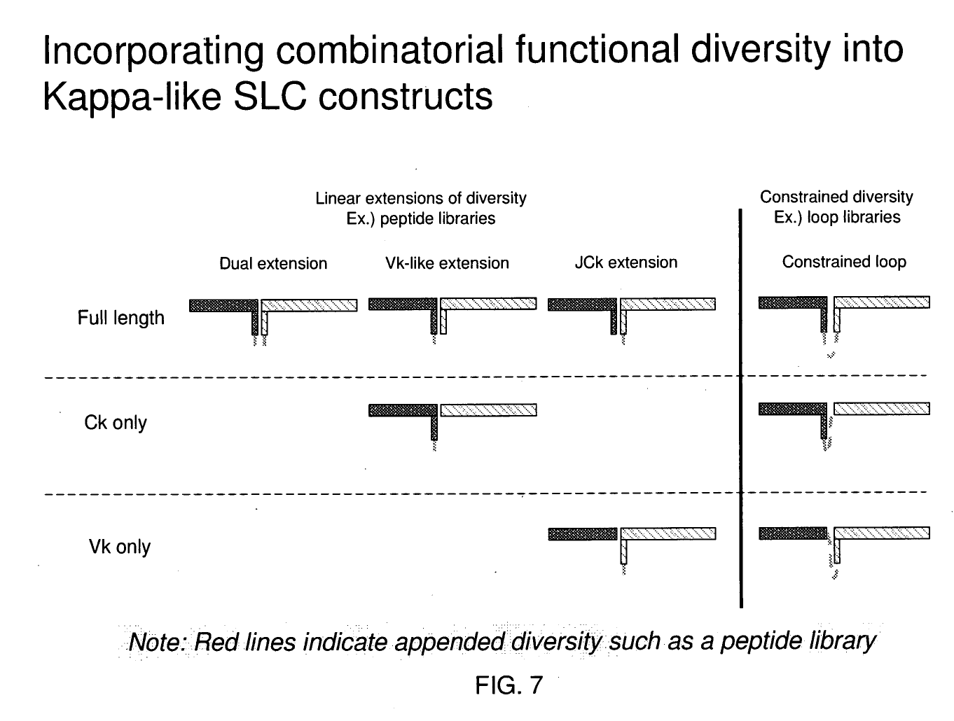

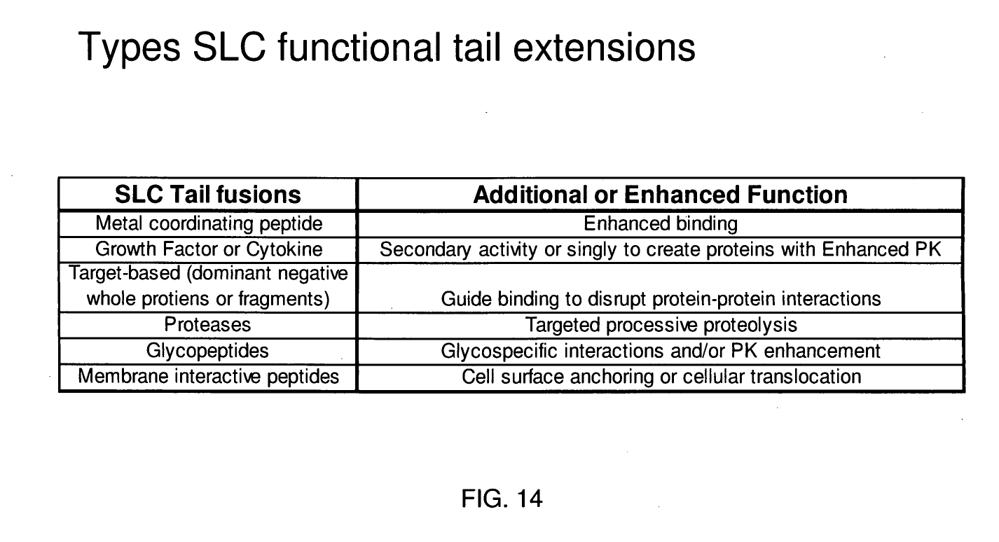

While the C- and N-terminal extensions of the Vκ-like polypeptide and/or the JCκ polypeptide do not need to be present in the constructs of the present invention, it is advantageous to retain at least a part of at least one of such appendages, because they provide a unique opportunity to create combinatorial functional diversity, either by linear extensions or, for example, in the form of constrained diversity, as a result of screening loop libraries, as shown in FIG. 7. In addition, the “tail” portions of the Vκ-like polypeptide and/or the JCκ polypeptide can be fused to other peptides and/or polypeptides, to provide for various desired properties, such as, for example, enhanced binding, additional binding specificities, enhanced pK, improved half-life, reduced half-life, cell surface anchoring, enhancement of cellular translocation, dominant negative activities, etc. Specific functional tail extensions are listed in FIG. 14.

If desired, the constructs of the present invention can be engineered, for example, by incorporating or appending known sequences or sequence motifs from the CDR1, CDR2 and/or CDR3 regions of antibodies, including known therapeutic antibodies into the CDR1, CDR2 and/or CDR3 analogous regions of the K-like surrogate light chain sequences. This allows the creation of molecules that are not antibodies, but will exhibit binding specificities and affinities similar to or superior over those of a known therapeutic antibody.

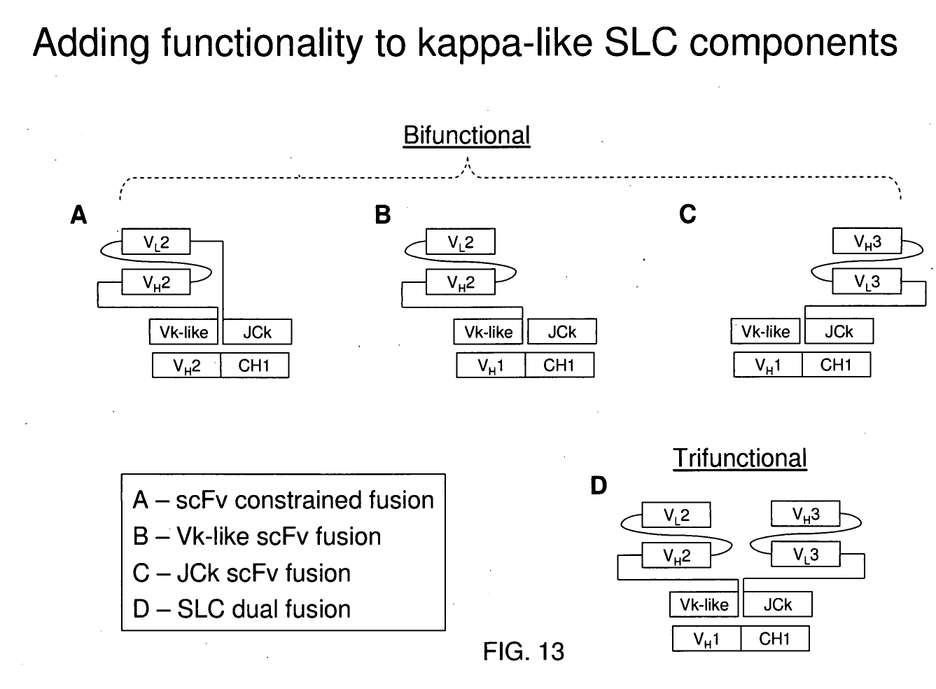

In certain embodiments, the heterogeneous amino acid sequence can add one or more additional functionalities to the construct of the present invention. Such constructs with additional functionalities including antibody variable region sequences with desired binding specificities are illustrated in FIG. 13. In particular, FIG. 13 illustrates a variety of bifunctional and trifunctional constructs, including Vκ-like and JCκ polypeptide sequences as hereinabove described.

While the constructs of the present invention are illustrated by reference to certain embodiments, one of ordinary skill will understand that numerous further embodiments obtained by various permutations of surrogate light chain and antibody sequences are possible, and are within the scope of the present invention. The present invention includes all constructs that comprise surrogate light chain sequences and have the ability to bind a desired target. In certain embodiment, the constructs also have the ability to associate with antibody heavy chain variable region sequences.

The constructs of the present invention may be used to build libraries of surrogate light chain sequences, which can be used for various purposes, similarly to antibody libraries, including selection of constructs with the desired binding specificities and affinities.

As Vκ-like and the JCκ genes encode polypeptides that can function as independent proteins and function as surrogate light chains, surrogate-like light chains can be engineered from true light chains and be used in every previous application proposed for engineered true surrogate light chains. This can be accomplished by expressing the variable light region to contain a peptidic extension analogous to either the VpreB or Vκ-like gene. Similarly the constant region can be engineered to resemble either the lambda 5 or JCκ genes and their peptidic extensions. Furthermore any chimeras or heterodimeric partnered combinations are within the scope herein.

Preparation of κ-like Surrogate Light Chain Constructs

The κ-like surrogate light chain constructs of the present invention can be prepared by methods known in the art, including well known techniques of recombinant DNA technology.

Nucleic acid encoding κ-like surrogate light chain polypeptides, can be isolated from natural sources, e.g. developing B cells and/or obtained by synthetic or semi-synthetic methods. Once this DNA has been identified and isolated or otherwise produced, it can be ligated into a replicable vector for further cloning or for expression.

Cloning and expression vectors that can be used for expressing the coding sequences of the polypeptides herein are well known in the art and are commercially available. The vector components generally include, but are not limited to, one or more of the following: a signal sequence, an origin of replication, one or more marker genes, an enhancer element, a promoter, and a transcription termination sequence. Suitable host cells for cloning or expressing the DNA encoding the surrogate light chain constructs in the vectors herein are prokaryote, yeast, or higher eukaryote (mammalian) cells, mammalian cells are being preferred.

Examples of suitable mammalian host cell lines include, without limitation, monkey kidney CV1 line transformed bySV40 (COS-7, ATCC CRL 1651); human embryonic kidney line 293 (293 cells) subcloned for growth in suspension culture, Graham et al, J. Gen Virol. 36:59 (1977)); baby hamster kidney cells (BHK, ATCC CCL 10); Chinese hamster ovary cells/-DHFR(CHO, Urlaub et al., Proc. Natl. Acad. Sci. USA 77:4216 (1980)); mouse sertoli cells (TM4, Mather, Biol. Reprod. 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TR1 cells (Mather et al., Annals N.Y. Acad. Sci. 383:44-68 (1982)); and MRC 5 cells; FS4 cells.

For use in mammalian cells, the control functions on the expression vectors are often provided by viral material. Thus, commonly used promoters can be derived from the genomes of polyoma, Adenovirus2, retroviruses, cytomegalovirus, and Simian Virus 40 (SV40). Other promoters, such as the β-actin protomer, originate from heterologous sources. Examples of suitable promoters include, without limitation, the early and late promoters of SV40 virus (Fiers et al., Nature, 273: 113 (1978)), the immediate early promoter of the human cytomegalovirus (Greenaway et al., Gene, 18: 355-360 (1982)), and promoter and/or control sequences normally associated with the desired gene sequence, provided such control sequences are compatible with the host cell system.

Transcription of a DNA encoding a desired heterologous polypeptide by higher eukaryotes is increased by inserting an enhancer sequence into the vector. The enhancer is a cis-acting element of DNA, usually about from 10 to 300 bp, that acts on a promoter to enhance its transcription-initiation activity. Enhancers are relatively orientation and position independent, but preferably are located upstream of the promoter sequence present in the expression vector. The enhancer might originate from the same source as the promoter, such as, for example, from a eukaryotic cell virus, e.g. the SV40 enhancer on the late side of the replication origin (bp 100-270), the cytomegalovirus early promoter enhancer, the polyoma enhancer on the late side of the replication origin, and adenovirus enhancers.

Expression vectors used in mammalian host cells also contain polyadenylation sites, such as those derived from viruses such as, e.g., the SV40 (early and late) or HBV.

An origin of replication may be provided either by construction of the vector to include an exogenous origin, such as may be derived from SV40 or other viral (e.g., Polyoma, Adeno, VSV, BPV) source, or may be provided by the host cell.

The expression vectors usually contain a selectable marker that encodes a protein necessary for the survival or growth of a host cell transformed with the vector. Examples of suitable selectable markers for mammalian cells include dihydrofolate reductase (DHFR), thymidine kinase (TK), and neomycin.

The transformed host cells may be cultured in a variety of media. Commercially available media include Ham's F10 (Sigma), Minimal Essential Medium ((MEM), (Sigma), RPMI-1640 (Sigma), and Dulbecco's Modified Eagle's Medium ((DMEM), Sigma). In addition, any of the media described in Ham et al., Meth. Enz. 58:44 (1979) and Barnes et al., Anal. Biochem. 102:255 (1980) may be used as culture media for the host cells. The culture conditions, such as temperature, pH, and the like, are those previously used with the host cell selected for expression, and are included in the manufacturer's instructions or will otherwise be apparent to the ordinarily skilled artisan.

Libraries Comprise κ-like Surrogate Light Chain Sequences

The present invention further concerns various libraries of κ-like surrogate light chain sequences and constructs comprising such sequences. Thus, such libraries may comprise, consist essentially of, or consist of, displays of κ-like surrogate light chain sequences, such as the Vκ-like and/or JCκ containing constructs of the present invention, including, without limitation, those specifically described above or in the examples.

The libraries of the present invention are preferably in the form of a display. Systems for displaying heterologous proteins, including antibodies and other polypeptides, are well known in the art. Antibody fragments have been displayed on the surface of filamentous phage that encode the antibody genes (Hoogenboom and Winter J. Mol. Biol., 222:381 388 (1992); McCafferty et al., Nature 348(6301):552 554 (1990); Griffiths et al. EMBO J., 13(14):3245-3260 (1994)). For a review of techniques for selecting and screening antibody libraries see, e.g., Hoogenboom, Nature Biotechnol. 23(9):1105-1116 (2005). In addition, there are systems known in the art for display of heterologous proteins and fragments thereof on the surface of Escherichia coli (Agterberg et al., Gene 88:37-45 (1990); Charbit et al., Gene 70:181-189 (1988); Francisco et al., Proc. Natl. Acad. Sci. USA 89:2713-2717 (1992)), and yeast, such as Saccharomyces cerevisiae (Boder and Wittrup, Nat. Biotechnol. 15:553-557 (1997); Kieke et al., Protein Eng. 10:1303-1310 (1997)). Other known display techniques include ribosome or mRNA display (Mattheakis et al., Proc. Natl. Acad. Sci. USA 91:9022-9026 (1994); Hanes and Pluckthun, Proc. Natl. Acad. Sci. USA 94:4937-4942 (1997)), DNA display (Yonezawa et al., Nucl. Acid Res. 31(19):e118 (2003)); microbial cell display, such as bacterial display (Georgiou et al., Nature Biotech. 15:29-34 (1997)), display on mammalian cells, spore display (Isticato et al., J. Bacteriol. 183:6294-6301 (2001); Cheng et al., Appl. Environ. Microbiol. 71:3337-3341 (2005), viral display, such as retroviral display (Urban et al., Nucleic Acids Res. 33:e35 (2005), display based on protein-DNA linkage (Odegrip et al., Proc. Acad. Natl. Sci. USA 101:2806-2810 (2004); Reiersen et al., Nucleic Acids Res. 33:e10 (2005)), and microbead display (Sepp et al., FEBS Lett. 532:455-458 (2002)).

For the purpose of the present invention, the surrogate light chain-containing libraries may be advantageously displayed using any display technique, including phage display and spore display.

In phage display, the heterologous protein, such as a surrogate light chain polypeptide, is linked to a coat protein of a phage particle, while the DNA sequence from which it was expressed is packaged within the phage coat. Details of the phage display methods can be found, for example, McCafferty et al., Nature 348, 552-553 (1990)), describing the production of human antibodies and antibody fragments in vitro, from immunoglobulin variable (V) domain gene repertoires from unimmunized donors. According to this technique, antibody V domain genes are cloned in-frame into either a major or minor coat protein gene of a filamentous bacteriophage, such as M13 or fd, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. Thus, the phage mimics some of the properties of the B-cell. Phage display can be performed in a variety of formats; for their review see, e.g. Johnson, Kevin S, and Chiswell, David J., Current Opinion in Structural Biology 3, 564-571 (1993). Several sources of V-gene segments can be used for phage display. Clackson et al., Nature 352, 624-628 (1991) isolated a diverse array of anti-oxazolone antibodies from a small random combinatorial library of V genes derived from the spleens of immunized mice. A repertoire of V genes from unimmunized human donors can be constructed and antibodies to a diverse array of antigens (including self-antigens) can be isolated essentially following the techniques described by Marks et al., J. Mol. Biol. 222, 581-597 (1991), or Griffith et al., EMBO J. 12, 725-734 (1993). In a natural immune response, antibody genes accumulate mutations at a high rate (somatic hypermutation). Some of the changes introduced will confer higher affinity, and B cells displaying high-affinity surface immunoglobulin are preferentially replicated and differentiated during subsequent antigen challenge. This natural process can be mimicked by employing the technique known as “chain shuffling” (Marks et al., Bio/Technol. 10, 779-783 (1992)). In this method, the affinity of “primary” human antibodies obtained by phage display can be improved by sequentially replacing the heavy and light chain V region genes with repertoires of naturally occurring variants (repertoires) of V domain genes obtained from unimmunized donors. This technique allows the production of antibodies and antibody fragments with affinities in the nM range. A strategy for making very large phage antibody repertoires has been described by Waterhouse et al., Nucl. Acids Res. 21, 2265-2266 (1993). These, and other techniques known in the art, can be adapted to the display of any polypeptide, including polypeptides and other constructs comprising surrogate light chain sequences.

Spore display, including surface display system using a component of the Bacillus subtilis spore coat (CotB) and Bacillus thuringiensis (Bt) spore display, is described in Isticato et al., J. Bacteriol. 183:6294-6301 (2001); Cheng et al., Appl. Environ. Microbiol. 71:3337-3341 (2005), the entire disclosures of which is hereby expressly incorporated by reference. Other spore display systems are disclosed in U.S. Patent Application Publication Nos. 20020150594; 20030165538; 20040180348; 20040171065; and 20040254364.

Uses of κ-like Surrogate Light Chain Sequences, Constructs and Libraries Containing Same

The libraries of the present invention can be used to identify κ-like surrogate light chain sequences and κ-like surrogate light chain constructs, such as fusions comprising surrogate light chain sequences, with desired properties. For example, in vitro or in vivo screening of the libraries herein can yield polypeptides comprising κ-like surrogate light chain sequences binding to desired targets with high binding specificity and affinity. Thus, the libraries herein can be used to identify molecules for therapeutic and diagnostic purposes, such as polypeptides comprising surrogate light chain sequences that bind to tumor markers or other molecular targets of therapeutic intervention. In addition, by the techniques described above, highly diverse libraries of surrogate light chain polypeptides can be engineered, including libraries comprising a collection of polypeptides binding to the same target, libraries of polypeptides binding to different targets, libraries of polypeptides with multiple specificities, and the like.

Further details of the invention are provided in the following non-limiting Examples.

### Example 1

### The κ-like SLC Components, Vκ-like or JCκ as Binding Domain Proteins

To make a Vκ-like binding domain, a single protein shown in FIGS. 2 and 4 (SEQ ID NO: 2) is created recombinantly. The surrogate light chain (SLC) binding domain protein construct is comprised of amino acids 21 to 180 (this may be as short as 3-6 amino acids) from the predicted secreted Vκ-like protein of SEQ ID NO: 2. If desired, to create novel and specific binding capabilities, the molecule is reengineered according to structural or sequence evidence. Reengineering may include, for example, CDRs and/or diversification within the C-terminal tail, through random or rationally designed mutagenesis means. Additionally, or alternatively, a collection of variants are created along the entire length of the Vκ-like protein either randomly, for example by error-prone PCR, or directly by single- or multi-site specific mutagenesis with a collection of amino acids. The resulting clones or collections can then be cloned in frame with pIII for use in phage or phagemid display. This phagemid constructs are then transformed into TG1 cells, propagated in Luria Broth (LB) supplemented with 50 μg/ml Ampicillin and 2% glucose until it reached OD600 ˜0.3, and infected with MK307 helper phage at 37° C. for 30 minutes without shaking The cells are pelleted and then resuspended in LB containing 50 μg/ml ampicillin and 75 μg/ml kanamycin and allowed to grow overnight with vigorous aeration at 30° C. The next day, the supernatant containing phagemid expressed Vκ-like proteins are panned against human TNF-α, according to commonly accepted panning methods. Specific Vκ-like clones that bind TNF-α, can be enriched through iterative selection and amplification and then individually assessed for binding, also utilizing commonly accepted m13 phage assessment methods, which typically involve either Phage ELISA or testing of crude or purified periplasmic lysates of the secreted proteins produced in E. coli.

The above description concerns the preparation of Vκ-like binding proteins, but the Vκ-like protein can also be recombinantly recombined with other heterologous sequences that recognize a common target and screened as a library. Furthermore, this Vκ-like binding protein can be combined with a previously selected collection of antibody heavy chains and screened directly on the same target of interest or a second target of interest to create a bispecific molecule. Alternatively, this reinforced binding or bispecific binding can be discovered by screening in conjunction with unselected collections of heavy chains.

While this example refers to antibody heavy chains, it should be understood that a complete heavy chain is not needed. Combinations comprising heavy chain variable region sequences, in the absence of a heavy chain constant region, or a complete heavy chain constant region, can be made in an analogous manner and are within the scope of this example.

Finally, JCκ can be made and used in the same manner, except that diversification could be incorporated in the N-terminal tail extension, non-CDR loops, or throughout the entire length of the JCκ protein, singularly or in the combinations mentioned above.

### Example 2

### Kappa SLC Construction

Coding sequences of the kappa surrogate light chain components of the heterodimeric SLC deletion variants shown in FIG. 5 (also referred to as “SURROBODY™ variants”) can be co-transfected with a full-length IgG1 antibody heavy chain into CHO-K1 cells (ATCC CCL-61) to transiently produce surrogate light chain constructs for biochemical analysis.

For example, full length Vκ-like (SEQ ID NO: 2) and JCκ (SEQ ID NO: 4) are cloned separately into the mammalian expression vector pCI (Promega, Madison Wis.). For both of these proteins portions of their predicted nonstructural tails are deleted. For Vκ-like this C-terminal tail follows the Kabat analogous residue #95 or residues 122-146 of SEQ ID NO: 2 and for JCκ this includes the J region N-terminal amino acids 1-28 of SEQ ID NO: 4. Specifically for JCκ the residues 1-28 represent the predicted start through the end of the kappa J-region. The Vκ-like constructs contain native predicted secretion signals and in the case of Vκ-like this predicted signal peptide is amino acids 1-20 of SEQ ID NO: 2. JCκ, however, does not appear to have a canonical signal sequence. Frances et. al. have shown that the JCκ can associate with surface exposed heavy chains, indicating that the translated protein is capable of transport or translocation to the extracellular surface. However, for overexpression purposes a set of variants are also created containing appended canonical mammalian light chain signal sequences to the amino terminus of the JCκ protein or to any tail deletion variants to improve SURROBODY™ protein production. The sequence of this truncated JCκ sequence is shown as SEQ ID NO: 24 and when combined with a heavy chain and full length Vκ-like forms the SLC deletion variant designated in FIG. 5 as “dJ”. The sequence of the truncated Vκ-like sequence is shown as SEQ ID NO: 25 and when combined with a heavy chain and full length JCκ forms the SLC deletion variant designated in FIG. 5 as “dVκ tail.” When both truncated JCκ sequence and truncated Vκ-like proteins are combined with a heavy chain the SLC deletion variant is designated in FIG. 5 as “short kappa.”

### Example 3

### Expression and Purification of Kappa Surrogate Light Chain Constructs (SURROBODY™) in Mammalian Cells

Each of the four combinatorial kappa surrogate light chain possibilities in FIG. 5 can be co-transfected with a known human anti-influenza virus heavy chain, containing a C-terminal hexahistidine (His6) tag (SEQ ID NO: 26), and expressed according to manufacturer's suggestions (Invitrogen, Carlsbad Calif.) in low serum media. After 3 days the supernatants are collected, filtered, and purified by nickel chelate chromatography (Qiagen, Germany). The purified proteins are then examined by western blot analysis with either anti-peptide rabbit serum (Vκ-like and JCκ) or anti-histidine antibodies (Serotec, Raleigh N.C.). Detection of proteins is visualized following anti-rabbit HRP (Vκ-like and JCκ) or anti-mouse HRP (heavy chain) and colorimetric development with TMB substrate.

Each of the combinatorial kappa surrogate light chain variants is tested for the ability to bind the cognate antigen related to the anti-influenza heavy chain. This can be performed either with purified proteins or clarified transfection supernatants. In any event, wells of a 96-well ELISA plate are coated and blocked with H5N1 hemagglutinin (Vietnam 1203) as described by Kashyap et al., Proc. Natl. Acad. Sci. 105:5986-5991 (2008). Next, the SURROBODIES™ are added and allowed to bind antigen for 1 hour at room temperature. Following a washing step with PBS+0.05% Tween, binding is then quantitatively detected by using anti-human Fc-HRP antibodies and TMB substrate colorimetrical detection recording absorbance readings at 450 nm.

### Example 4

### Adding Functionalities to Kappa SLC Components

As the kappa SLC is comprised of two independent polypeptides this creates natural opportunities to append or embed secondary functionalities. In the present Example, in the first instance an anti-VEGF scFv is inserted to create a fusion protein linking Vκ-like, or dVκ tail and either JCκ or dJ (FIG. 13A). The resulting engineered kappa SLC-constrained scFvs are paired with the heavy chain of an anti-TNF-α antibody. The desired protein is produced by co-transfecting the individual constrained fusions with the full length heavy chain, containing a C-terminal hexahistidine (His6) tag (SEQ ID NO: 27), and expressing the proteins according to manufacturer's suggestions (Invitrogen, Carlsbad Calif.) in low serum media. After 3 days the secreted SURROBODIES™ are collected from the media, filtered, and purified by nickel chelate chromatography (Qiagen, Germany).

The resulting protein is used in ELISA to determine targeted binding. Briefly, the ELISA entails coating and blocking of an ELISA plate with human TNF-α or human VEGF, followed by incubation of the kappa SLC SURROBODIES™ for 2 hours at 4° C., washing with PBS-Tween-20 (0.05%) and direct detection with anti-human heavy chain-HRP antibody.

Alternatively the fusion of the anti-VEGF scFv to the C-terminus of Vκ-like (FIG. 13B) or to the N-terminus of JCκ (FIG. 13C) can be made, and the resulting protein complex construct assessed similarly to the surrobody ELISA described above.

Finally, fusion of the anti-VEGF scFv to the C-terminus of Vκ-like and an anti-ovalbumin scFv is fused to the amino terminus of JCκ and the tripartite protein complex tested for binding to VEGF, TNF-α, and ovalbumin (FIG. 13D).

In the description scFv, against disparate targets are incorporated, however one can combine functional binders to the same target to create tandem “super-binders.” These tandem binders can either provide reinforced binding or even in some instances cross-linking function. Fab cross-linking will be beneficial in instances where whole antibodies provide undesirable and prolonged cross-linking For instance, it may be undesirable for whole immunoglobulin insulin receptor antibodies that act as insulin substitutes to require 3-4 weeks for serum clearance. As insulin usually has a half-life of minutes, a Fab would be more in tune with this scale of half-life and the tandem functionality could appropriately address this application.

The above descriptions describe only antibodies as secondary functional groups, but one can also similarly incorporate relevant peptides (e.g., erythropoietin mimetics), receptors (e.g., TNF-RI), dominant negative whole proteins (e.g., DN-TNF, Steed et al., Science. 301:1895-1898 (2003)) antagonistic fragments or domains (e.g., HGF-based NK1 or NK4 domains) and binding proteins (e.g., IL-1ra) to the appended and constrained constructs to create molecules of similar functions. Also one might utilize the two sites to incorporate heterodimeric proteins, such as heavy and light chains to create a secondary Fab-like molecule.

### Example 5

### Kappa SLC Libraries

As described Vκ-like and JCκ domains can be used as single binding entities, but they can also be combined with heavy chains to produce combinatorial libraries for panning against antigens. The heavy chains may be nave or hyperimmunized lymphocyte derived collections or synthetic collections. In some instances it may be beneficial to have heavy chain collections from previously enriched antibody libraries used in combination with kappa SLC libraries. In any case, a fully diverse collection of kappa SLC SURROBODIES™ can, under appropriate selection and design as described above in Example 1, provide multiple antigen selectivity through independent binding elements in each kappa SLC component, or provide enhanced binding through additional binding, not existing in classical antibodies, against target through the Vκ-like and JCκ tails.

Specifically we will use an iterative approach using combinatorial antibody libraries prepared from the bone marrow of H5N1 avian influenza survivors. These libraries are screened against H5N1 viral hemagglutinin protein for two rounds of selection. Next, the phagemid plasmids are amplified, purified, and the heavy chain variable regions isolated by restriction digest from this plasmid preparation. These heavy chains are then be cloned in frame with the constant heavy domain 1 to form a recombinant fusion to the m13 gene III coat protein for phagemid display.

Following panning, ELISA test is conducted for clonal antigen binding phage from all appropriate rounds of selection and libraries by transferring enriched clones into the HB2151 E. coli strain to produce soluble SURROBODY™ proteins. Briefly, HB2151 clones will be grown and induced to produce soluble SURROBODIES™. Specifically, colonies are cultured overnight in 2-YT media supplemented with 100 mcg/ml ampicillin and 200 micromolar IPTG overnight at 30 degrees and the periplasmic lysates, as described above, tested by ELISA, essentially as outlined previously.

### Example 6

### Engineering SLC-like Molecules from Existing Light Chain V Genes and Light Chain Constant Genes

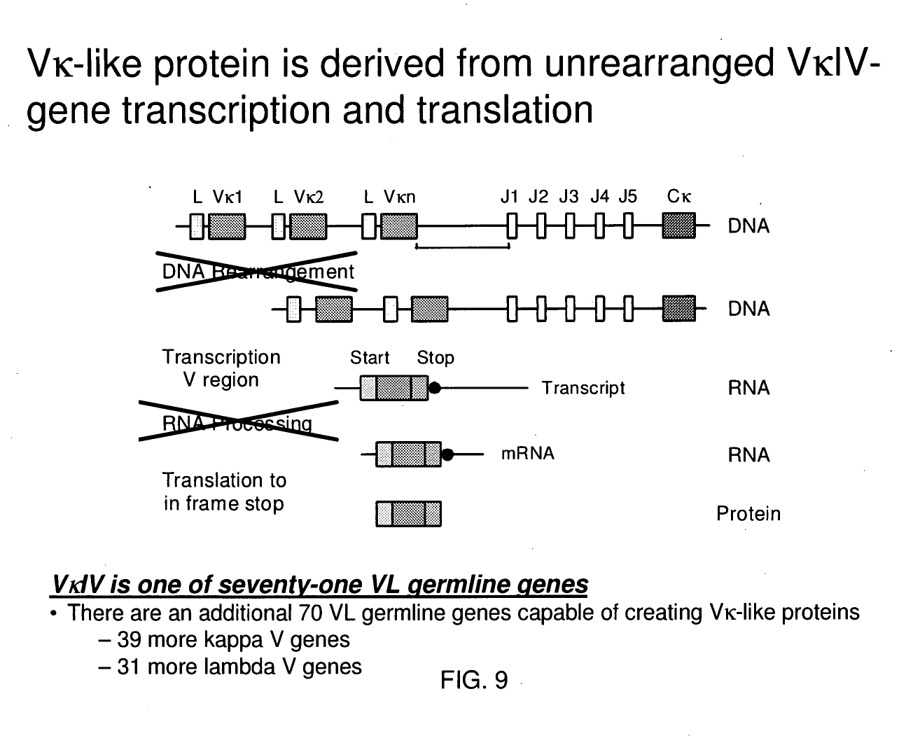

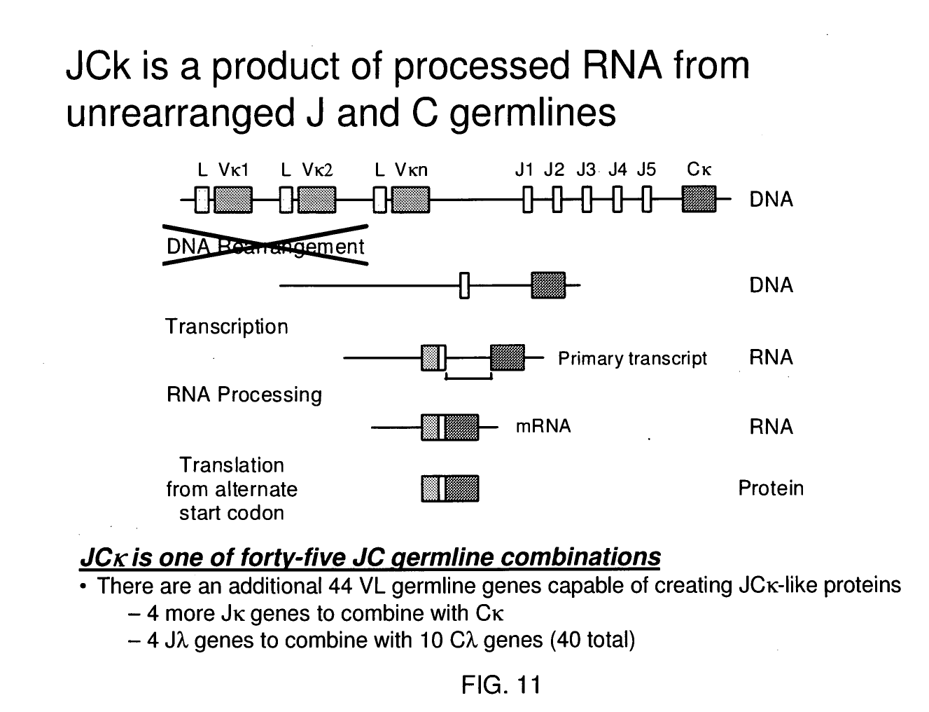

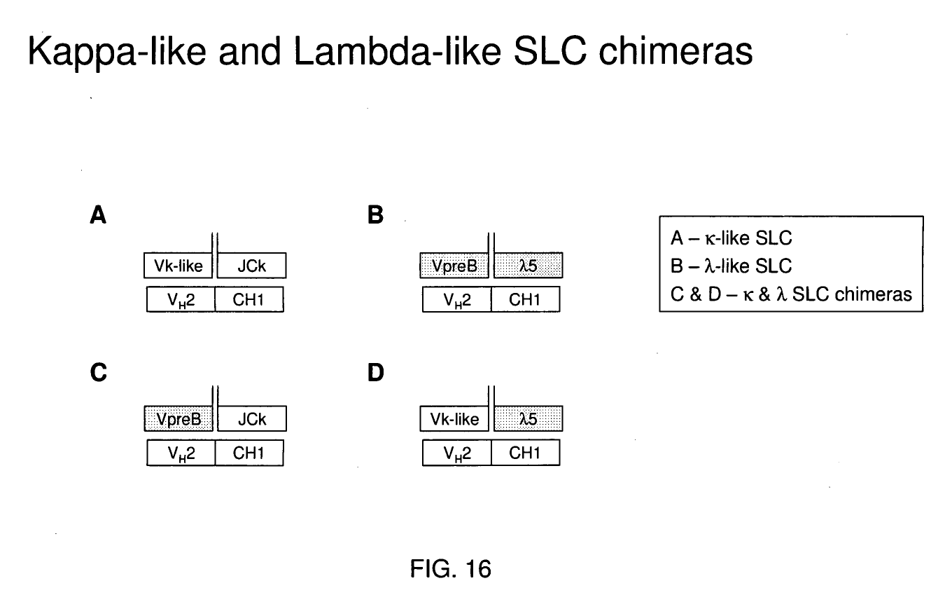

As the components of the kappa SLC provide alternative function from unrearranged light chain V genes and rearranged light chain JC genes, it is feasible to engineer similar translated proteins from all remaining kappa and lambda light chain V genes to make Vκ-like molecules (FIGS. 9 and 10) and all combinations of the remaining kappa JC rearrangements (4 JCκ-like) (FIGS. 11 and 12) and lambda JC rearrangements (4 “J”×10 “constant”=40 JCλ-like) (FIG. 11). Each one of these engineered molecules can serve purposes similar to those using Vκ-like and JCκ, as well as those contained in co-pending PCT application Serial No. PCT/US2008/058283, filed on Mar. 28, 2007, with VpreB and λ5, and combinations and chimeras thereof. (FIG. 16)

### Example 7

### Kappa Surrogate Light Chain Fusions to Increase Serum Half-life

The half-life of an antibody fragment in vivo is extended considerably when it is part of a fusion to an intact and complete heavy chain that includes all heavy chain constant domains, not just those necessary to form a stable antigen binding fragment. In the case of IgG this means the inclusion of domains CH1, CH2, and optionally CH3. In particular it is well established that CH2 and CH3 confer the majority of this effect in vivo. In fact fusion of these CH2 and CH3 domains to heterologous proteins is typically sufficient to improve the potencies and PK/PD of these chimeric molecules compared to the parent molecules. Similarly functional fusions to either or both Vκ-like and JCκ benefit by this association with the constant domains of the heavy chain.

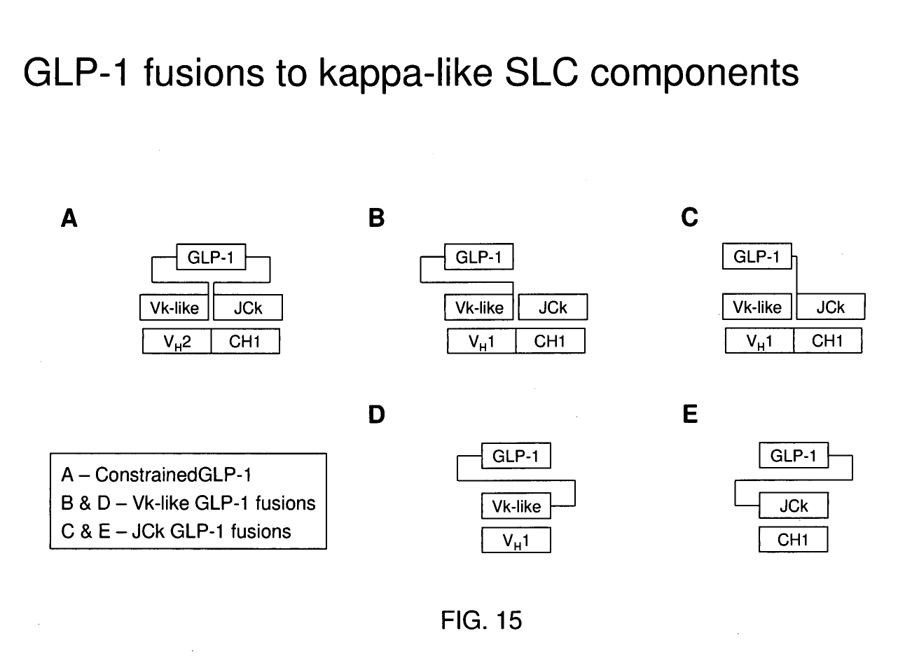

For the treatment of type II diabetes administration of glucagon-like peptide 1 (or GLP-1) benefits individuals by inducing glucose-dependent insulin secretion in the pancreas, thereby improving glucose management in those patients. However, a long-lived GLP-1 peptide is a desirable goal. As the tails of the kappa surrogate light chains are distinct and accessible, this goal can be accomplished by either recombinantly fusing the active GLP-1 moiety to either the C-terminus of the Vκ-like tail (SEQ ID NO: 28) or the N-terminal tail of JCκ (SEQ ID NO: 29). In the case of a JCκ fusion expression may be performed in the presence or absence of Vκ-like and even in the presence or absence of the variable heavy domain, as depicted in FIG. 15. Fusions to Vκ-like can similarly be made in the presence or absence of JCκ, and possibly with or without the CH1 domain of the heavy chain. Similarly, other beneficial growth factor, cytokine, receptor, and enzyme fusions may be created. In all of these cases binding is not requisite of the surrogate light chain, or SURROBODY™ components, but rather may be conferred either entirely or in large part by the heterologous surrogate light chain fused element.

Although in the foregoing description the invention is illustrated with reference to certain embodiments, it is not so limited. Indeed, various modifications of the invention in addition to those shown and described herein will become apparent to those skilled in the art from the foregoing description and fall within the scope of the appended claims.

What is claimed is:

1. A κ-like surrogate light chain (SLC) construct comprising a Vκ-like and/or a JCκ sequence.

2. The κ-like SLC construct of claim 1 comprising a Vκ-like sequence.

3. The κ-like SLC construct of claim 1 comprising a JCκ sequence.

4. The κ-like SLC construct of claim 1 comprising both a Vκ-like sequence and a JCκ sequence.

5. The κ-like SLC construct of claim 1 capable of specifically binding to a target.

6. The κ-like SLC construct of claim 5 wherein the Vκ-like sequence comprises SEQ ID NO: 2, with or without a signal sequence and with or without a C-terminal tail, or a fragment thereof.

7. The κ-like SLC construct of claim 6 wherein the Vκ-like sequence comprises the N-terminal signal peptide (amino acids 1-20) of SEQ ID NO: 2.

8. The κ-like SLC construct of claim 7 wherein the Vκ-like sequence comprises at least part of the C-terminal tail from within SEQ ID NO: 2.

9. The κ-like SLC construct of claim 5 wherein the Vκ-like sequence is selected from the group comprising SEQ ID NOs: 7-18, with or without a signal sequence and with or without a C-terminal tail, or a fragment thereof.

10. The κ-like SLC construct of claim 5 wherein the JCκ sequence comprises SEQ ID NO: 4, with or without an N-terminal extension, or a fragment thereof.

11. The κ-like SLC construct of claim 5 wherein the JCκ sequence comprises a sequence selected from the group consisting of SEQ ID NOs: 19-23, with or without an N-terminal extensions, or a fragment thereof.

12. The κ-like SLC construct of any one of claims 1-3 and 5-11 associated with an antibody heavy chain sequence.

13. The κ-like SLC construct of claim 4 associated with an antibody heavy chain sequence.

14. The κ-like SLC construct of claim 13 wherein the Vκ-like sequence comprises a C-terminal tail.

15. The κ-like SLC construct of claim 13 wherein the JCκ sequence comprises an N-terminal extension

16. The κ-like SLC construct of claim 13 wherein the Vκ-like sequence comprises a C-terminal tail and the JCκ sequence comprises an N-terminal extension.

17. The κ-like SLC construct of claim 13 wherein the Vκ-like sequence is devoid of a C-terminal tail and the JCκ sequence is devoid of an N-terminal extension.

18. The κ-like SLC construct of claim 12 wherein the antibody heavy chain sequence is a full-length antibody heavy chain or a fragment thereof.

19. The κ-like SLC construct of claim 13 wherein the antibody heavy chain sequence is a full-length antibody heavy chain or a fragment thereof.

20. The κ-like SLC construct of claim 13 wherein the Vκ-like sequence and the JCκ sequence are covalently linked to each other.

21. The κ-like SLC construct of claim 20 wherein the linkage is direct fusion.

22. The κ-like SLC construct of claim 20 wherein the linkage is through a heterogenous linker

23. The κ-like SLC construct of claim 22 wherein the heterogenous linker comprises a sequence of a native polypeptide or a fragment thereof.

24. The κ-like SLC construct of claim 22 wherein the heterogenous linker comprises a sequence of a therapeutic polypeptide or a fragment thereof.

25. The κ-like SLC construct of claim 22 wherein the heterogenous linker comprises an antibody sequence.

26. The κ-like SLC construct of claim 25 wherein the antibody sequence comprises antibody light chain and heavy chain variable region sequences.

27. The κ-like SLC construct of claim 26 wherein the antibody light chain and heavy chain sequences are capable of binding an antigen.

28. The κ-like SLC construct of claim 27 wherein the antigen is different from the target to which said construct binds.

29. The κ-like SLC construct of claim 13 comprising at least one antigen binding region of an antibody covalently linked to the Vκ-like sequence and/or the JCκ sequence.

30. The κ-like SLC construct of claim 29 which is bifunctional.

31. The κ-like SLC construct of claim 29 which is trifunctional.

32. The κ-like SLC construct of claim 1 wherein the Vκ-like sequence comprises a C-terminal tail and the JCκ sequence comprises an N-terminal extension.

33. The κ-like SLC construct of claim 32 wherein the C-terminal tail and/or the N-terminal extension is linked to a heterogeneous molecule.

34. The κ-like SLC construct of claim 33 wherein the heterogeneous molecule is a peptide or a polypeptide.

35. The κ-like SLC construct of claim 12, having improved pharmacokinetic profile relative to an antibody with the same binding specificity.

36. The κ-like SLC construct of claim 12 having improved potency relative to an antibody with the same binding specificity.

37. The κ-like SLC construct of claim 12 having improved specificity relative to an antibody binding to the same target.

38. A library comprising a collection of the κ-like SLC constructs of claim 1.

39. A library of claim 38 in the form of a display.

40. The library of claim 39 wherein said display is selected from the group consisting of phage display, bacterial display, yeast display, ribosome display, mRNA display, DNA display, display on mammalian cells, spore display, viral display, display based on protein-DNA linkage, and microbead display.

41. The library of claim 40 wherein the display is phage display.

42. The library of claim 38 further comprising a collection of antibody sequences.

43. The library of claim 42 wherein the antibody sequences comprise heavy and/or light chain variable region sequences.

44. The library of claim 38 comprising a collection of Vκ-like sequences.

45. The library of claim 44 wherein said collection of Vκ-like sequences comprises Vκ-like sequence variants that differ in their CDR sequences and/or in the C-terminal sequences.

46. The library of claim 38 comprising a collection of JCκ sequences.

47. The library of claim 46 wherein said collection of JCκ sequences comprises JCκ sequence variants that differ in their N-terminal extensions.
